(19) United States
(12) Patent Application Publication
(10) Pub. No.: US 2011/0117113 A1
(43) Pub. Date: May 19, 2011

(54) IMMUNOGLOBULIN SINGLE VARIABLE DOMAIN DIRECTED AGAINST HUMAN CXCR4 AND OTHER CELL ASSOCIATED PROTEINS AND METHODS TO GENERATE THEM

(76) Inventors: Gerald Beste, Gent (BE); Stephanie Staelens, Wevelgem (BE); Peter Vanlandschoot, Bellem (BE); Maria Gonzalez Pajuelo, Porto (PT); Hilde Adi Pierrette Revets, Meise (BE); Peter Schotte, De Pinte (BE); Hilde Stals, Gent (BE); Ann Brige, Ertvelde (BE); Maarten Dewilde, Gent (BE); Catelijne Stortelers, Gent (BE)

(21) Appl. No.: 12/897,051

(22) Filed: Oct. 4, 2010

Related U.S. Application Data
(60) Provisional application No. 61/250,264, filed on Oct. 9, 2009.
(60) Provisional application No. 61/358,495, filed on Jun. 25, 2010.

(51) Int. Cl. A61K 39/395; C07K 16/28; C07K 16/46; C07K 19/00; A61P 35/00; A61P 35/02; A61P 35/04; A61P 29/00; A61P 31/00; A61P 17/06; A61P 11/06; A61P 11/00; A61P 37/06

(52) U.S. Cl. 424/173.1; 530/389.6; 530/387.3; 530/387.9

(57) ABSTRACT

The invention relates to immunoglobulin single variable domains directed against specific human CXCR4 epitopes (herein also referred to interchangeably as “compounds of the invention”, “amino acid sequences of the invention”, or “building blocks of the invention”) and polypeptides comprising them (herein also referred to as “polypeptides of the invention”, “compounds of the invention”, or “constructs of the invention”).

Furthermore, the present invention relates to nucleic acids encoding the compounds of the invention (also referred to herein as “nucleic acids of the invention” or “nucleotide sequences of the invention”); to methods for preparing the compounds of the invention; to host cells expressing or capable of expressing the compounds of the invention; to compositions, and in particular to pharmaceutical compositions, that comprise the compounds of the invention; and to uses of the compounds of the invention and the aforementioned nucleic acids, host cells and/or compositions, in particular for prophylactic, therapeutic or diagnostic purposes, such as the prophylactic, therapeutic or diagnostic purposes mentioned herein.

The invention also relates to methods for generating immunoglobulin single variable domains against a target such as a cell-associated protein and constructs comprising said immunoglobulin single variable domains. The invention also provides immunoglobulin single variable domains obtainable by the methods of the invention. Specifically, the present invention relates to the generation of immunoglobulin single variable domains and constructs thereof by use of epitope walking with multimer libraries. More specifically, the present invention relates to the generation of immunoglobulin single variable domains derived from camelids directed against a particular epitope of a target, in particular against a target with multiple transmembrane spanning domains, including GPCRs and ion channels, by epitope walking with multimer libraries.

### RELATED APPLICATIONS

This application claims the benefit under 35 U.S.C. §119(e) of U.S. provisional application No. 61/250,264, filed Oct. 9, 2009, and of U.S. provisional application No. 61/358,495, filed Jun. 25, 2010, the disclosures of each of which is incorporated by reference herein.

### FIELD OF THE INVENTION

The invention relates to immunoglobulin single variable domains directed against specific human CXCR4 epitopes (herein also referred to interchangeably as “compounds of the invention”, “amino acid sequences of the invention”, or “building blocks of the invention”) and polypeptides comprising them (herein also referred to as “polypeptides of the invention”, “compounds of the invention”, or “constructs of the invention”).

Furthermore, the present invention relates to nucleic acids encoding the compounds of the invention (also referred to herein as “nucleic acids of the invention” or “nucleotide sequences of the invention”); to methods for preparing the compounds of the invention; to host cells expressing or capable of expressing the compounds of the invention; to compositions, and in particular to pharmaceutical compositions, that comprise the compounds of the invention; and to uses of the compounds of the invention and the aforementioned nucleic acids, host cells and/or compositions, in particular for prophylactic, therapeutic or diagnostic purposes, such as the prophylactic, therapeutic or diagnostic purposes mentioned herein.

The invention also relates to methods for generating immunoglobulin single variable domains against a target such as a cell-associated protein and constructs comprising said immunoglobulin single variable domains. The invention also provides immunoglobulin single variable domains obtainable by the methods of the invention. Specifically, the present invention relates to the generation of immunoglobulin single variable domains and constructs thereof by use of epitope walking with multimer libraries. More specifically, the present invention relates to the generation of immunoglobulin single variable domains derived from camelids directed against a particular epitope of a target, in particular against a target with multiple transmembrane spanning domains, including GPCRs and ion channels, by epitope walking with multimer libraries.

### BACKGROUND OF THE INVENTION

The International patent application with publication number WO2009/138519 (entitled “AMINO ACID SEQUENCES DIRECTED AGAINST CXCR4 AND OTHER GPCRs AND COMPOUNDS COMPRISING THE SAME” and published on 19 Nov. 2009) describes amino acid sequences (such as immunoglobulin single variable domains including domain antibodies, single domain antibodies, dAb's, VHH's and Nanobodies®) that are directed against CXCR4 and other GPCRs. The teaching of WO 2009/138519 is incorporated herein by reference.

One aspect of WO 2009/138519 relates to amino acid sequences such as immunoglobulin single variable domains that are directed against and specific for CXCR4 and in particular against human CXCR4. For example, in one specific aspect, WO 2009/138519 describes “multivalent” (as defined in WO 2009/138519), and “multispecific” (as defined in WO 2009/138519) such as e.g. bivalent constructs that are directed against CXCR4. Some non-limiting examples thereof are the anti-CXCR4 constructs described in Example 4 and Example 5.

Immunoglobulin single variable domains, such as antibodies and antigen binding fragments derived therefrom are widely used to specifically target their respective antigens in research and therapeutic applications. Typically, the generation of antibodies involves the immunization of experimental animals, fusion of antibody producing cells to create hybridomas and screening for the desired specificities. Alternatively, antibodies can be generated by screening of immune, naïve or synthetic libraries e.g. by phage display.

The generation of immunoglobulin single variable domains, such as Nanobodies, has been described extensively in various publications, among which WO 94/04678 is herein exemplified. In these methods, camelids are immunized with the target antigen in order to induce an immune response against said target antigen. The repertoire of immunoglobulin single variable domains obtained from said immunization is further screened for immunoglobulin single variable domains that bind the target antigen.

### SUMMARY OF THE INVENTION

An important class of potential therapeutic targets, to which it is difficult to obtain binding molecules, are cell associated antigens, including transmembrane antigens, in particular transmembrane antigens with multiple membrane spanning domains. Desired epitopes, e.g. epitopes that when targeted give rise to yet unknown agonistic, antagonistic, non-functional or inverse agonistic activity, of such cell-associated, and especially membrane bound antigens, however, are usually difficult to target by antibodies and thus the generation of antibodies and fragments thereof with conventional techniques such as immunization and subsequent screening as e.g. described in WO 94/04678 have often been not successful.

In one aspect, the applicant has now identified some particularly preferred immunoglobulin single variable domains and classes of monovalent, multispecific (such as bispecific) and multivalent (such as in particular bi- and/or trivalent—as herein defined) compounds that are directed against CXCR4 and in particular to human CXCR4. In doing so, applicant has also identified some particularly preferred binding interactions and epitopes on CXCR4, and in particular for human CXCR4 for (monovalent, multivalent, multispecific and/or multivalent and multispecific) compounds that bind to CXCR4 and in particular to human CXCR4. The data to these particularly preferred binding interactions and epitopes on human CXCR4 optionally in combinations with the corresponding compounds can now be used to generate other immunoglobulin single variable domains with the same or similar particularly preferred binding interactions and epitopes on human CXCR4 by methods know to skilled person in the art. Thus, the current invention provides further immunoglobulin single variable domains with the same or similar particularly preferred binding interactions and epitopes on human CXCR4 as the now identified classes of mulspecific or/and multivalent constructs. In some of these aspects, immunoglobulin single variable domains and constructs thereof do not include the compounds with clone names 238D2 (SEQ ID NO: 2), 238D4 (SEQ ID NO: 3) and polypeptides comprising one of 238D2 and/or 238D4 as disclosed in WO2009/138519.

In another aspect, the present invention provides methods of inhibiting biological process wherein CXCR4 is involved and/or implicated. Furthermore, the invention provides methods for identifying modulators of CXCR4.

Furthermore, the art provides no satisfactory and efficient methods to generate immunoglobulin single variable domains against a new epitope of a target, in particular of a target that is a membrane associated protein, starting from identified such as the herein particularly preferred classes of monovalent, multispecific and/or multivalent compounds directed to human CXCR4.

Thus, it is the objective of the present invention to overcome these shortcomings of the art. In particular it is an objective of the present invention to provide i) immunoglobulin single variable domains with the same or similar particularly preferred binding interactions and epitopes on human CXCR4 as the now identified classes of monovalent, mulspecific or/and multivalent compounds; and ii) a method for creating immunoglobulin single variable domains against particular novel epitopes to complex antigens, like cell associated antigens.

In one embodiment of the invention, the immunoglobulin single variable domains are light chain variable domain sequences (e.g. a V_{L}-sequence), or heavy chain variable domain sequences (e.g. a V_{H}-sequence); more specifically, the immunoglobulin single variable domains can be heavy chain variable domain sequences that are derived from a conventional four-chain antibody or heavy chain variable domain sequences that are derived from a heavy chain antibody.

According to the invention, the immunoglobulin single variable domains can be domain antibodies, or amino acid sequences that are suitable for use as domain antibodies, single domain antibodies, or amino acid sequences that are suitable for use as single domain antibodies, “dAbs”, or amino acid sequences that are suitable for use as dAbs, or Nanobodies, including but not limited to V_{HH} sequences, and preferably are Nanobodies or V_{HH} sequences.

### BRIEF DESCRIPTION OF THE FIGURES

FIG. 1 provides an overview of the generation of Nanobody-fusion phage libraries with different orientations. (A) first immunoglobulin single variable domain known to bind antigen is in the N-terminal position, (B) second immunoglobulin single variable domain selected from set, collection or library is in the N-terminal position.

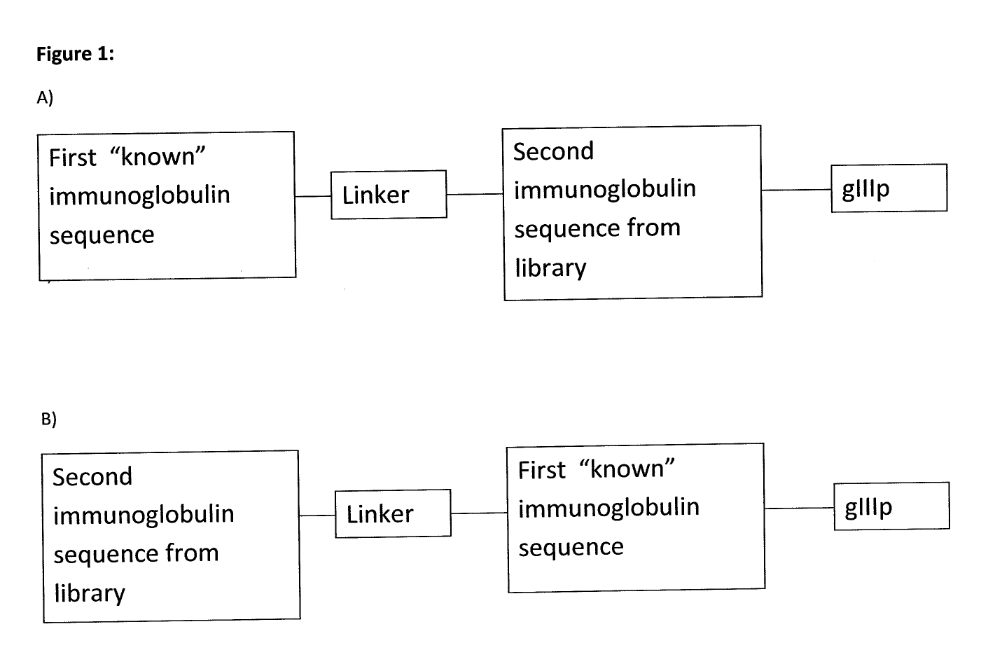

FIG. 2 shows selected vector constructs of the invention.

FIG. 3 shows the critical Residues of 238D2 and 238D4 mapped onto CXCR4 (SEQ ID NO: 6). The critical residues for 283D4 are S178, E179, D187. The critical residues for 283D2 are P191, N192, W195, V196 and E277. F189 is a critical residue for both 283D4 and 283D2.

### DETAILED DESCRIPTION OF THE INVENTION

The present invention encompasses, but is not limited to, the subject matter of the appended claims.

### 1.) Definitions and General Methods

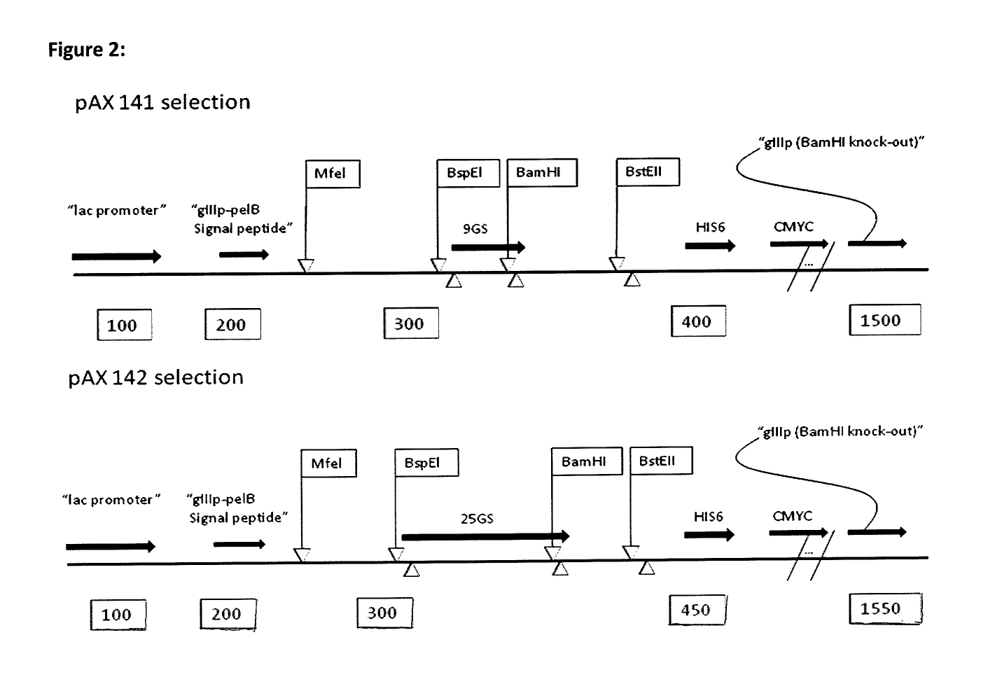

a) Unless indicated or defined otherwise, all terms used have their usual meaning in the art, which will be clear to the skilled person. Reference is for example made to the standard handbooks mentioned in paragraph a) on page 46 of WO 08/020,079.
b) Unless indicated otherwise, the term “immunoglobulin single variable domain” is used as a general term to include but not limited to antigen-binding domains or fragments such as V_{HH} domains or V_{H} or V_{L} domains, respectively. The terms antigen-binding molecules or antigen-binding protein are used interchangeably and include also the term nanobodies. The immunoglobulin single variable domains further are light chain variable domain sequences (e.g. a V_{L}-sequence), or heavy chain variable domain sequences (e.g. a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be domain antibodies, or immunoglobulin sequences that are suitable for use as domain antibodies, single domain antibodies, or immunoglobulin sequences that are suitable for use as single domain antibodies, “dAbs”, or immunoglobulin sequences that are suitable for use as dAbs, or nanobodies, including but not limited to V_{HH} sequences. The invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. The immunoglobulin single variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1″or “CDR1”; as “Complementarity Determining Region 2” or “CDR2”; and as “Complementarity Determining Region 3” or “CDR3”, respectively. It is noted that the terms nanobody or nanobodies are registered trademarks of Ablynx N.V. and thus may also be referred to as Nanobody® and/or Nanobodies®).
c) Unless indicated otherwise, the terms “immunoglobulin sequence”, “sequence”, “nucleotide sequence” and “nucleic acid” are as described in paragraph b) on page 46 of WO 08/020,079.
d) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2 (1): 49-57; Irving et al., J. Immunol. Methods, 2001, 248 (1-2), 31-45; Schmitz et al., Placenta, 2000, 21 Suppl. A, S106-12, Gonzales et al., Tumour Biol., 2005, 26 (1), 31-43, which describe techniques for protein engineering, such as affinity maturation and other techniques for improving the specificity and other desired properties of proteins such as immunoglobulins.
e) Amino acid residues will be indicated according to the standard three-letter or one-letter amino acid code. Reference is made to Table A-2 on page 48 of the International application WO 08/020,079 of Ablynx N.V. entitled “Immunoglobulin single variable domains directed against IL-6R and polypeptides comprising the same for the treatment of diseases and disorders associated with Il-6 mediated signalling”.
f) For the purposes of comparing two or more nucleotide sequences, the percentage of “sequence identity” between a first nucleotide sequence and a second nucleotide sequence may be calculated or determined as described in paragraph e) on page 49 of WO 08/020,079 (incorporated herein by reference), such as by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position); or using a suitable computer algorithm or technique, again as described in paragraph e) on pages 49 of WO 08/020,079 (incorporated herein by reference).
g) For the purposes of comparing two or more immunoglobulin single variable domains or other amino acid sequences such e.g. the polypeptides of the invention etc, the percentage of “sequence identity” between a first amino acid sequence and a second amino acid sequence (also referred to herein as “amino acid identity”) may be calculated or determined as described in paragraph f) on pages 49 and 50 of WO 08/020,079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein; or using a suitable computer algorithm or technique, again as described in paragraph f) on pages 49 and 50 of WO 08/020,079 (incorporated herein by reference).
   Also, in determining the degree of sequence identity between two immunoglobulin single variable domains, the skilled person may take into account so-called “conservative” amino acid substitutions, as described on page 50 of WO 08/020,079. Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J. Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
h) Immunoglobulin single variable domains and nucleic acid sequences are said to be “exactly the same” if they have 100% sequence identity (as defined herein) over their entire length.
i) When comparing two immunoglobulin single variable domains, the term “amino acid difference” refers to an insertion, deletion or substitution of a single amino acid residue on a position of the first sequence, compared to the second sequence; it being understood that two immunoglobulin single variable domains can contain one, two or more such amino acid differences.
j) When a nucleotide sequence or amino acid sequence is said to “comprise” another nucleotide sequence or amino acid sequence, respectively, or to “essentially consist of” another nucleotide sequence or amino acid sequence, this has the meaning given in paragraph i) on pages 51-52 of WO 08/020,079.
k) The term “in essentially isolated form” has the meaning given to it in paragraph j) on pages 52 and 53 of WO 08/020,079.
l) The terms “domain” and “binding domain” have the meanings given to it in paragraph k) on page 53 of WO 08/020,079.
m) The terms “antigenic determinant” and “epitope”, which may also be used interchangeably herein, have the meanings given to it in paragraph l) on page 53 of WO 08/020,079.
n) As further described in paragraph m) on page 53 of WO 08/020,079, an amino acid sequence (such as an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
o) The term “specificity” has the meaning given to it in paragraph n) on pages 53-56 of WO 08/020,079; and as mentioned therein refers to the number of different types of antigens or antigenic determinants to which a particular antigen-binding molecule or antigen-binding protein (such as a polypeptide of the invention) molecule can bind. The specificity of an antigen-binding protein can be determined based on affinity and/or avidity, as described on pages 53-56 of WO 08/020,079 (incorporated herein by reference), which also describes some preferred techniques for measuring binding between an antigen-binding molecule (such as a polypeptide of the invention) and the pertinent antigen. Typically, antigen-binding proteins (such as the immunoglobulin to single variable domains, and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin single variable domain of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. As will be clear to the skilled person, and as described on pages 53-56 of WO 08/020,079, the dissociation constant may be the actual or apparent dissociation constant. Methods for determining the dissociation constant will be clear to the skilled person, and for example include the techniques mentioned on pages 53-56 of WO 08/020,079.
p) The half-life of an amino acid sequence, compound or polypeptide of the invention can generally be defined as described in paragraph o) on page 57 of WO 08/020,079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 08/020,079. As also mentioned in paragraph o) on page 57 of WO 08/020,079, the half-life can be expressed using paraMeters such as the t1/2-alpha, t1/2-beta and the area under the curve (AUC). Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The terms “increase in half-life” or “increased half-life” as also as defined in paragraph o) on page 57 of WO 08/020,079 and in particular refer to an increase in the t1/2-beta, either with or without an increase in the t1/2-alpha and/or the AUC or both.
q) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
r) An immunoglobulin single variable domain or polypeptide is said to be “specific for” a first target or antigen compared to a second target or antigen when is binds to the first antigen with an affinity/avidity (as described above, and suitably expressed as a K_{D} value, K_{A} value, K_{off} rate and/or Kₒₙ rate) that is at least 10 times, such as at least 100 times, and preferably at least 1000 times, and up to 10.000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10.000 times less or even less than that, than the K_{D} with which said amino acid sequence or polypeptide binds to the second target or polypeptide. Preferably, when an immunoglobulin single variable domain or polypeptide is “specific for” a first target or antigen compared to a second target or antigen, it is directed against (as defined herein) said first target or antigen, but not directed against said second target or antigen.
s) The terms “cross-block”, “cross-blocked” and “cross-blocking” are used interchangeably herein to mean the ability of an immunoglobulin single variable domain or polypeptide to interfere with the binding directly or indirectly through allosteric modulation of other immunoglobulin single variable domains or polypeptides of the invention to a given target. The extend to which an immunoglobulin single variable domain or polypeptide of the invention is able to interfere with the binding of another to target, and therefore whether it can be said to cross-block according to the invention, can be determined using competition binding assays. One particularly suitable quantitative cross-blocking assay uses a FACS- or an ELISA-based approach to measure competition between the labelled (e.g. His tagged or radioactive labelled) immunoglobulin single variable domain or polypeptide according to the invention and the other binding agent in terms of their binding to the target. The experimental part generally describes suitable FACS-, ELISA- or radioligand-displacement-based assays for determining whether a binding molecule cross-blocks or is capable of cross-blocking an immunoglobulin single variable domain or polypeptide according to the invention. It will be appreciated that the assay can be used with any of the immunoglobulin single variable domains or other binding agents described herein. Thus, in general, a cross-blocking amino acid sequence or other binding agent according to the invention is for example one which will bind to the target in the above cross-blocking assay such that, during the assay and in the presence of a second amino acid sequence or other binding agent of the invention, the recorded displacement of the immunoglobulin single variable domain or polypeptide according to the invention is between 60% and 100% (e.g. in ELISA/radioligand based competition assay) or between 80% to 100% (e.g. in FACS based competition assay) of the maximum theoretical displacement (e.g. displacement by cold (e.g. unlabeled) immunoglobulin single variable domain or polypeptide that needs to be cross-blocked) by the to be tested potentially cross-blocking agent that is present in an amount of 0.01 mM or less (cross-blocking agent may be another conventional monoclonal antibody such as IgG, classic monovalent antibody fragments (Fab, scFv)) and engineered variants (diabodies, triabodies, minibodies, VHHs, dAbs, VHs, VLs).
t) An amino acid sequence such as e.g. an immunoglobulin single variable domain or polypeptide according to the invention is said to be “cross-reactive” for two different antigens or antigenic determinants (such as serum albumin from two different species of mammal, such as human serum albumin and cyno serum albumin) if it is specific for (as defined herein) both these different antigens or antigenic determinants.
u) As further described in paragraph q) on pages 58 and 59 of WO 08/020,079 (incorporated herein by reference), the amino acid residues of an immunoglobulin single variable domain are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication), and accordingly FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.
v) An amino acid sequence is said to be “an epitope A binder” if said amino acid sequence i) is an immunoglobulin single variable domain such as a VHH (e.g. Nanobody); and ii) displaces to 90%, more preferably 95%, most preferred 99% 238D2 (SEQ ID NO: 2) at an immunoglobulin single variable domain concentration below 100 nM in a displacement assay such as shown in the experimental part; and iii) does not specifically bind (or only to a limited extend) to CXR4 mutant 1 (F189V; SEQ ID NO: 19) and to CXCR4 mutant 2 (V196E; SEQ ID NO: 20) at 10 nM, 30 nM or 100 nM immunoglobulin single variable domain concentration in the so called footprint assay as shown in the experimental part. However these compounds specifically bind to CXCR4 mutant 3 (D187V; SEQ ID NO: 21) very similarly as the wild type human CXCR4 at 10 nM, 30 nM or 100 nM immunoglobulin single variable domain concentration again as tested in the so called footprint analysis as shown in the experimental part.
w) An amino acid sequence is said to be “an epitope B binder” if said amino acid sequence i) is an immunoglobulin single variable domain such as a VHH (e.g. Nanobody); and ii) displaces to 90%, more preferably 95%, most preferred 99% 238D4 (SEQ ID NO: 3) at an immunoglobulin single variable domain concentration below 100 nM in a displacement assay such as shown in the experimental part; and iii) does not specifically bind (or only to a limited extend) to CXR4 mutant 1 (F189V; SEQ ID NO: 19) and to CXCR4 mutant 3 (D187V; SEQ ID NO: 21) at 10 nM, 30 nM or 100 nM immunoglobulin single variable domain concentration in the so called footprint assay as shown in the experimental part. However these compounds specifically bind CXCR4 mutant 2 (V196E; SEQ ID NO: 20) very similarly as the wild type human CXCR4 at 10 nM, 30 nM or 100 nM immunoglobulin single variable domain concentration again as tested in the so called footprint assay as shown in the experimental part.
x) Human CXCR4 occurs naturally in 2 alternative spliced forms, i.e. isoform 1 (SEQ ID NO: 6) and isoform 2 (SEQ ID NO: 22)—Uniprot information from 10 Aug. 2010.
y) The Figures, Sequence Listing and the Experimental Part/Examples are only given to further illustrate the invention and should not be interpreted or construed as limiting the scope of the invention and/or of the appended claims in any way, unless explicitly indicated otherwise herein.

Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e,g, of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].

The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as an amino acid sequence, immunoglobulin single variable domain, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RTln(K_{D}) (equivalently DG=−RTln(K_{A})), where R equals the gas constant, T equals the absolute temperature and ln denotes the natural logarithm.

The K_{D} for biological interactions, such as the binding of the immunoglobulin single variable domains of the invention to the cell associated antigen as defined herein, which are considered meaningful (e.g. specific) are typically in the range of 10⁻¹⁰M (0.1 nM) to 10⁻⁵M (10000 nM). The stronger an interaction is, the lower is its K_{D}.

The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹.

As regards immunoglobulin single variable domains of the invention, the on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate of immunoglobulin single variable domains of the invention may vary between 10⁻⁶ s⁻¹ (near irreversible complex with a t_{1/2} of multiple days) to 1 s⁻¹ (t_{1/2}=0.69 s).

The affinity of a molecular interaction between two molecules can be measured via different techniques known per se, such as the well known surface plasmon resonance (SPR) biosensor technique (see for example Ober et al., Intern Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known Biacore instruments.

It will also be clear to the skilled person that the measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule. In such situation the measured affinity may be affected by the avidity of the interaction by the two molecules.

Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic.

However, the accurate measurement of K_{D} may be quite labour-intensive and as consequence, often apparent K_{D} values are determined to assess the binding strength of two molecules. It should be noted that as long as all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance.

Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorbance detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+C_{ref}K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.

In the context of the present invention, “conformation dependent epitope”, or “conformational epitope” denotes an epitope that comprises amino acids which are not within a single consecutive stretch of the primary sequence of the antigen. In other words, due to the secondary and/or tertiary structure of a protein target, amino acids which may be spaced apart in the primary sequence are brought into proximity to each other and thereby participate in the formation of an epitope. If for example an antigen comprises three amino acid loops, residues on each one of these loops may participate in the formation of a single epitope. The same applies to antigens comprising more than one domain or subunit. In this case, an epitope may be formed by amino acids on different domains or subunits. Complete or partial denaturing of the protein by appropriate conditions, i.e. the partial or full destruction of secondary and/or tertiary structures, will also partly or fully destroy conformational epitopes. The skilled person will understand that the precise conditions under which a conformational epitope is destroyed by denaturing a protein will depend on the nature of the protein and the specific circumstances.

In a preferred embodiment, the present invention is directed to immunoglobulin single variable domains against conformational epitopes. In particular, the invention concerns immunoglobulin single variable domains against conformational epitopes on cell-associated antigens as defined herein, which may preferably be camelid immunoglobulin single variable domains, including Nanobodies.

In the context of the present invention, “cell-associated antigen” means antigens that are firmly anchored in or located within the membranes of a cell (including membranes of subcellular compartments and organelles), and includes antigens that have a single or multiple transmembrane regions. In other words, the term refers to antigens exhibiting membrane-dependent conformational epitopes. In particular, the term refers to antigens having conformational epitopes as defined herein. The term encompasses transmembrane antigens with a single membrane-spanning region and transmembrane antigens with multiple membrane spanning domains such as GPCRs or ion channels, and preferably encompasses transmembrane antigens with multiple membrane spanning domains. Amongst all these antigens the skilled person knows a range of druggable target antigens, which represent a preferred cell associated antigen of the present invention. The invention in particular relates to cell associated antigens wherein the conformation dependent epitope is dependent on the correct anchoring and/or location in the membrane. Thus, the invention provides immunoglobulin single variable domains against such conformation dependent epitopes.

In a preferred embodiment the invention relates to antigens that are integral membrane proteins having one, or more preferably multiple membrane spanning domains. These antigens will reside in and operate within a cell's plasma membrane, and/or the membranes of subcellular compartments and organelles. Many transmembrane proteins, such as transmembrane receptors comprise two or more subunits or domains, which functionally interact with one another.

Integral membrane proteins comprise three distinct parts or domains, i.e. an extracellular (or extracompartmental) domain, a transmembrane domain and an intracellular (or intracompartmental) domain. A protein having multiple transmembrane domains will typically also have multiple extra- and intra cellular/compartmental domains. For example, a seven transmembrane receptor will comprise seven transmembrane domains.

Thus, the term cell associated antigen as understood herein is intended to exclude antigens that are only loosely associated, i.e. that are not firmly anchored or located within a membrane. An antigen is firmly anchored if it comprises at least one domain or part that extends into the membrane.

In one embodiment, the invention excludes antigens that have a membrane insertion via a lipid tail, but no transmembrane domain. In this instance, the conformation of the hydrophilic portion or domain of the protein will not depend on the membrane environment. It will, for example, be possible to express a recombinant protein lacking the lipid tail, which is in the proper conformation, i.e. expresses the conformational epitopes also present if the antigen is associated with the membrane via the lipid tail. Similarly, any other proteins which are only loosely associated are excluded from the invention in a particular embodiment. “Loosely associated” in this connection means proteins which exhibit their natural conformation even in the absence of membrane, i.e. their natural conformation is not dependent on the anchoring or embedding within a membrane.

Typical examples of cell associated antigens according to the invention comprise seven membrane domain receptors, including G-protein coupled receptors, such as Adrenergic receptor, Olfactory receptors, Receptor tyrosine kinases, such as Epidermal growth factor receptor, Insulin Receptor, Fibroblast growth factor receptors, High affinity neurotrophin receptors, and Eph Receptors, Integrins, Low Affinity Nerve Growth Factor Receptor, NMDA receptor, Several Immune receptors including Toll-like receptor, T cell receptor and CD28.

As used herein, the term “cell-associated antigen” is intended to include, and also refer to, any part, fragment, subunit, or domain of said cell associated antigen. Any subsection of the cell associated antigen falls within the scope of the present invention, provided it represents a conformational epitope of interest. If for example the epitope of interest is located in a binding site of a receptor, or the pore of an ion channel, any fragment(s) of the cell associated antigen capable of forming said epitope are included in the invention. Preferably, those parts, domains, fragments or subunits will be those parts of the cell associated antigen, which are responsible for the membrane-dependent conformation. If for example a protein comprises several transmembrane domains, linked by extended intracellular loops, it is envisaged that such loops are in part or fully omitted, without influencing the extracellular conformational epitopes.

In particular, the present invention relates to immunoglobulin single variable domains directed to cell associated antigens in their natural conformation. In the context of the present invention, “natural conformation” means that the protein exhibits its secondary and/or tertiary structure, in particular its membrane dependent secondary and/or tertiary structure. In other words, the natural conformation describes the protein in a non-denatured form, and describes a conformation wherein the conformational epitopes, in particular the membrane dependent conformational epitopes, are present. Specifically, the protein will have the conformation that is present when the protein is integrated into or firmly attached to a membrane. Antigens can be obtained in their natural conformation when present in cells comprising natural or transfected cells expressing the cell-associated antigen, cell derived membrane extracts, vesicles or any other membrane derivative harbouring antigen, liposomes, or virus particles expressing the cell associated antigen. In any of these embodiments, antigen may be enriched by suitable means. Said cell-associated antigen can be expressed on any suitable cell allowing expression of the antigen in its native or natural conformation, encompassing, but not limited to Cho, Cos7, Hek293, or cells of camelid origin.

The cell associated antigen of the present invention is preferably a druggable membrane protein, in particular a druggable membrane protein having multiple membrane spanning domains. In one embodiment of the invention, the target is a GPCR or an ion channel.

Specific, non limiting examples of ion channels that represent cell associated antigens according to the present invention are provided in the following. Also listed are therapeutic effects of immunoglobulin single variable domains specifically recognizing such ion channels.

1. Two-P potassium channels (see Goldstein et al., Pharmacological
Reviews, 57, 4, 527 (2005)), such as K_{2P}1.1, K_{2P}2.1, K_{2P}3.1, K_{2P}3.1,
K_{2P}4.1, K_{2P}5.1, K_{2P}6.1, K_{2P}7.1, K_{2P}9.1, K_{2P}10.1, K_{2P}12.1, K_{2P}13.1,
K_{2P}15.1, K_{2P}16.1, K_{2P}17.1 and K_{2P}18.1, which can all be screened using
electrophysiological assays such as FLIPR or patch-clamp.
2. CatSper channels (see Clapham and Garbers, Pharmacological
Reviews, 57, 4, 451 (2005)), such as CatSper-1 and CatSper-2 (both
involved in fertility and sperm motility), CatSper-3 and CatSper-4,
which can all be screened using electrophysiological assays such as
FLIPR, patch-clamp or calcium imaging techniques.
3. Two-pore channels (see Clapham and Garbers, Pharmacological
Reviews, 57, 4, 451 (2005)), such as TPC1 and TPC2.
4. Cyclic nucleotide-gated channels (see Hofman et al.,
Pharmacological Reviews, 57, 4, 455 (2005), such as CNGA-1, CNGA-
2, CNGA-3, CNGA-4A, CNGB1 and CNGB3, which can be screened
using techniques such as patch-clamp and calcium imaging
5. Hyperpolarization-activated cyclic nucleotide-gated channels (see
Hofman et al., Pharmacological Reviews, 57, 4, 455 (2005)), such as
HCN1, HCN2, HCN3, HCN4 (all regarded as promising
pharmacological targets for development of drugs for cardiac
arrhythmias and ischemic heart disease), which can be screened using
techniques such as voltage-clamp.
6. Inwardly rectifying potassium channels (see Kubo et al.,
Pharmacological Reviews, 57, 4, 509 (2005)), such as Kᵢᵣ1.1, Kᵢᵣ21.
Kᵢᵣ2.2, Kᵢᵣ2.3, Kᵢᵣ2.4, Kᵢᵣ3.1, Kᵢᵣ3.2, Kᵢᵣ3.3, Kᵢᵣ3.4, Kᵢᵣ3.4, Kᵢᵣ4.2, Kᵢᵣ5.1,
Kᵢᵣ6.1 (a target for antihypertensive agents and coronary vasodilators),
Kᵢᵣ6.2 (the target for pentholamine; its subunit SUR1 is a target for the
treatment of diabetes and PHHI) and Kir7.1 (which is a possible site for
side-effects of calcium channel blockers), which can be screened using
techniques such as voltage-clamp.
7. Calcium-activated potassium channels (see Wei et al.,
Pharmacological Reviews, 57, 4, 463 (2005)), such as
K_{Ca}1.1 - openers of which may be useful in the treatment of stroke,
epilepsy, bladder over-reactivity, asthma, hypertension, gastric
hypermotility and psychoses;
K_{Ca}2.1 - modulators of which may be useful in the treatment of various
diseases such as myotonic muscular dystrophy, gastrointestinal
dysmotility, memory disorders, epilepsy, narcolepsy and alcohol
intoxication. Openers of
K_{Ca}2.2 have been proposed for cerebellar ataxia;
K_{Ca}2.2 - modulators of which may be useful in the treatment of various
diseases such as myotonic muscular dystrophy, gastrointestinal
dysmotility, memory disorders, epilepsy, narcolepsy and alcohol
intoxication. Openers of
K_{Ca}2.2 have been proposed for cerebellar ataxia;
K_{Ca}2.2 - modulators of which may be useful in the treatment of various
diseases such as myotonic muscular dystrophy, gastrointestinal
dysmotility, memory disorders, epilepsy, narcolepsy, hypertension and
urinary incontinence;
K_{Ca}3.1 - blockers of which may be useful in the treatment of sickle cell
anemia, diarrhea, as immunosuppressants, EAE, the prevention of
restenosis and angiogenesis, the treatment of brain injuries and the
reduction of brain oedema.
Openers if K_{Ca}3.1 have been proposed for the treatment of cystic
fibrosis and COPD; as well as K_{Ca}4.1, K_{Ca}4.2 and K_{Ca}5.1; all of which
can be screened using electrophysiological techniques or techniques
such as patch-clamp or voltage-clamp.
8. Potassium channels (see Shieh et al., Pharmacological Reviews, 57,
4, 557 (2005) and Gutman et al., Pharmacological Reviews, 57, 4, 473
(2005)), including:
voltage-gated calcium channels such as Kv1.1, Kv1.2, Kv1.3, Kv1.4,
Kv1.5, Kv1.6 and Kv.17;
voltage- and cGMP-gated calcium channels such as Kv1.10;
beta-subunits of Kv channels such as KvBeta-1, KvBeta-2 and KvBeta-3;
Shab-like channels such as Kv2.1 and Kv2.2;
Shaw-like channels such as Kv3.1, Kv3.2. Kv3.3 and Kv3.4;
Shal-like channels such as Kv4.1, Kv4.2, Kv4.3, Kv5.1, Kv6.1, Kv6.2,
Kv8.1, Kv9.1, Kv9.2, Kv9.3, KH1 and KH2;
Ether-a-go-go-channels such as EAG, HERG, BEC1 and BEC2;
MinK-type channels such as MinK, MiRP1 and MiRP2;
KvLQT -type channels such as KvLQT1, KvLQT2, KvLQT3, KvLQT4,
KvLQT5
Inwardly rectifying potassium channels such as those mentioned above;
Sulfonylurea receptors such as the sulfonylurea receptors 1 and 2;
Large conductance calcium-activated channels such as Slo and the Beta-
subunits of BK_{Ca};
Small conductance calcium-activated channels such as SK1, SK2 and
SK3;
Intermediate conductance calcium-activated channels such as IKCa1;
Two-pore potassium channels such as TWIK1, TREK, TASK, TASK2,
TWIK2, TOSS, TRAAK and CTBAK1;
all of which can be screened using electrophysiological techniques or
techniques such as patch-clamp or voltage-clamp. Potassium channels are
implicated in a wide variety of diseases and disorders such as cardiac
diseases (such as arrhythmia), neuronal diseases, neuromuscular
disorders, hearing and vestibular diseases, renal diseases, Alzheimer's
disease. and metabolic diseases; and are targets for active compounds in
these diseases. Reference is again made to the reviews by Shieh et al. and
by Gutman et al. (and the further prior art cited therein) as well as to the
further references cited in the present specification. Tables 3 and 4 of the
Shieh review also mention a number of known openers and blockers,
respectively, of various potassium channels and the disease indications
for which they have been used/proposed.
9. Voltage-gated calcium channels (see Catterall et al., Pharmacological
Reviews, 57, 4, 411 (2005)), such as:
Caᵥ1.2 - modulators of which are useful as Ca²⁺ antagonists;
Caᵥ1.3 - modulators of which have been proposed for modulating the
heart rate, as antidepressants and as drugs for hearing disorders;
Caᵥ2.1 - modulators of which have been proposed as analgesics for
inflammatory pain;
Caᵥ2.2 - - modulators of which have been proposed as analgesics for pain
such as inflammatory pain, postsurgical pain, thermal hyperalgesia,
chronic pain and mechanical allodynia;
Caᵥ3.2- which has been proposed as a target for epilepsy, hypertension
and angina pectoris;
Caᵥ3.3 - which has been proposed as a target for the treatment of thalamic
oscillations; and Caᵥ1.1, Caᵥ1.4, Caᵥ2.3, Caᵥ3.1,; all of which can be
screened using techniques such as patch-clamp, voltage-clamp and
calcium imaging.
10. Transient receptor potential (TRP) channels (see Clapham et al.,
Pharmacological Reviews, 57, 4, 427 (2005)) such as:
TRPC channels such as TRPC1, TRPC2, TRPC3, TRPC4, TRPC5,
TRPC6 and TRPC7;
TRPV channels such as TRPV1, TRPV2, TRPV3, TRPV4, TRPV5 and
TRPV6;
TRPM channels such as TRPM1, TRPM2, TRPM3, TRPM4, TRPM5,
TRPM6, TRPM7 and TRPM8;
TRPA1;
TRPP channels such as PKD1, , PKD2L1 and PKD2L2, which are
involved in polycystic kidney disease;
TRPML channels such as mucolipin 1, mucolipin 2 and mucolipin 3;
which can be screened using techniques such as patch-clamp and calcium
imaging.
11. Voltage-gated sodium channels (see Catterall et al., Pharmacological
Reviews, 57, 4, 397 (2005)), such as:
Naᵥ1.1, Naᵥ1.2 and Naᵥ1.3 - which are a target for drugs for the
prevention and treatment of epilepsy and seizures;
Naᵥ1.4 - which is a target for local anaesthetics for the treatment of
myotonia;
Naᵥ1.5 - which is a target for antiarrhythmic drugs;
Naᵥ1.6 - which is a target for antiepileptic and analgesic drugs;
Naᵥ1.7, Naᵥ1.8 and Naᵥ1.9 - which are potential targets for local
anaesthetics; all of which can be screened using voltage clamp or
techniques involving voltage-sensitive dyes.

Specific, non limiting examples of GPCRs that represent cell associated antigens according to the present invention are provided in the following. Also listed are some exemplary therapeutic effects of immunoglobulin single variable domains of the present invention that are directed against these GPCRs.

GPCRs are involved in a wide area variety of physiological processes. Some examples of their physiological roles include:

| | |
|---|---|
| 1. | Behavioral and mood regulation: receptors in the |
| | mammalian brain bind several different neurotransmitters, |
| | including serotonin, dopamine, GABA, and glutamate |
| 2. | Regulation of immune system activity and inflammation: |
| | chemokine receptors including CC chemokine receptor |
| | and/or CXC chemokine receptors bind ligands that |
| | mediate intercellular communication between cells of the |
| | immune system; receptors such as histamine receptors |
| | bind inflammatory mediators and engage target cell types in |
| | the inflammatory response |
| 3. | Autonomic nervous system transmission: both the |
| | sympathetic and parasympathetic nervous systems are |
| | regulated by GPCR pathways, responsible for control of |
| | many automatic functions of the body such as blood |
| | pressure, heart rate, and digestive processes |
| 4. | The visual sense: the opsins use a photoisomerization |
| | reaction to translate electromagnetic radiation into cellular |
| | signals |
| 5. | The sense of smell: receptors of the olfactory epithelium |
| | bind odorants (olfactory receptors) and pheromones |
| | (vomeronasal receptors) |

Preferably, said cell-associated antigen is a membrane-spanning antigen, including but not limited to an antigen selected from CXCR4. The skilled person will appreciate that there may be different specific three dimensional conformations that are encompassed by the term “natural conformation”. If, for example, a protein has two or more different conformations whilst being in a membrane environment, all these conformations will be considered “natural conformations”. This is exemplified by receptors changing their conformation by activation, e.g. the different activation states of rhodopsin induced by light, or ion channels showing a “closed” or “open” conformation. The invention encompasses immunoglobulin single variable domains to any one of these different natural conformations, i.e. to the different kinds of conformational epitopes that may be present.

A “nucleic acid” of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated form, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the cell associated antigen or immunoglobulin single variable domains of the invention, and/or can be isolated from a suitable natural source. To provide analogs, nucleotide sequences encoding naturally occurring V_{HH} domains can for example be subjected to site-directed mutagenesis, so as to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”. The genetic constructs of the invention may be DNA or RNA.

### 2) Immunoglobulin Single Variable Domains Directed Against Particular Epitopes of Human CXCR4

In a first aspect, the present invention relates to an immunoglobulin single variable domain that specifically binds to the second extracellular loop of CXCR4 and in particular to human CXCR4 (SEQ ID NO: 6, isoform 1 or SEQ ID NO: 22, isoform 2), i.e. said immunoglobulin single variable domain of this first aspect does not bind to the N-terminal part, nor the first and third extracellular loop of CXCR4.

In a second aspect, said immunoglobulin single variable domain of this first aspect has i) a maximal displacement of human CXCL12 (SDF-1) on human CXCR4 expressing HEK293T cells by more than 90% and ii) a receptor affinity Ki of 10 nM to said human CXCR4 expressing HEK293T cells (as e.g. measured in Example 1.6 of WO2009/138519).

In a third aspect, the present invention relates to an immunoglobulin single variable domain that specifically binds to one of the two particular epitopes, i.e.:
a) said immunoglobulin single variable domain specifically binds to epitope A that comprises and/or essentially consists of the conformation dependent epitope with the amino acid residues F189, N192, W195, P191, V196 and optionally E277, wherein the numbering of the amino acid residues refers to the human CXCR4-short sequence (SEQ ID NO: 6), herein also referred to as “epitope A binder” (see also definition of epitope A binder as defined herein); or
b) said immunoglobulin single variable domain specifically binds to epitope B that comprises and/or essentially consists of the conformation dependent epitope with the amino acid residues D187, F189, E179 and S178, wherein the numbering of the amino acid residues refers to the human CXCR4-short sequence (SEQ ID NO: 6), herein also referred to as “epitope B binder” (see also definition of epitope B binder as defined herein).

In a forth aspect, said immunoglobulin single variable domain of the above third aspect has i) a maximal displacement of human CXCL12 (SDF-1) on human CXCR4 expressing HEK293T cells by more than 90% and ii) a receptor affinity Ki of 10 nM or lower to said human CXCR4 expressing HEK293T cells (as e.g. measured in Example 1.6 of WO2009/138519).

In a fifth aspect, an immunoglobulin single variable domain as described herein (e.g. an immunoglobulin single variable domain of aspects one to four above) is not a compound with a sequence selected from the group consisting of 238D2 (SEQ ID NO: 2) or 238D4 (SEQ ID NO: 3) as also disclosed in WO2009/138519 under the same clone names.

For example, such epitope A binder may for example, and without limitation, be a variant of 238D2 and comprise one or more (further) “humanizing” substitutions (as defined herein) and/or comprise one or more of the following substitutions, compared to the sequence of 238D2:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from WO 09/068,627, which mention the various Camelid residues that occur as each amino acid position in wild-type VHH's). Such substitutions may even comprise suitable substitutions of an amino acid residue that occurs at a Hallmark position with another amino acid residue that occurding at a Hallmark position in a wild-type VHH (for which reference is for example made to Tables A-6 to A-9 from WO 09/068,627); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific examples of suitable substitutions (see for example pages 4 and 5 and pages 10 to 15). One example of such substitution may be to replace an NS motif at positions 82a and 82b with an NN motif;
   or any suitable combination of two or more of any of the foregoing substitutions (a) to (c).

For the purposes described herein, a humanizing substitution can generally be defined as a substitution whereby an amino acid residue that occurs in a framework regions of a camelid V_{HH} domain is replaced by a different amino acid that occurs at the same position in the framework region of a human V_{H} domain (and preferably, a human V_{H}3 domain). Thus, suitable humanizing substitutions will be clear to the skilled person based on the disclosure herein, the disclosure in WO 09/068,627, and from a comparison of the amino acid sequence of a given V_{HH} sequence and one or more human V_{H} sequences.

Reference is for example made to the Tables A-6 to A-9 of WO 09/068,627, which list some of the amino acid residues that have been found to occur in the framework regions of camelid VHH domains, and the corresponding amino acid residue(s) that most often occur in the framework regions of a human V_{H}3 sequence (such as for example, the germline sequences DP-47, DP-51 or DP-29). The humanizing substitutions that can be taken from these Figures are also some of the preferred humanizing substitutions used in the invention; however, it may also be possible to use humanizing substitutions that have been obtained by comparison with other germline sequences (from the V_{H}3 class or sometimes also from other V_{H} classes). As generally known from WO 09/068,627 (and from the patent applications from Applicant and the further prior art mentioned in WO 09/068,627), based on such sequence comparison, particularly suited and/or optimal humanizing substitutions (and combinations thereof) may generally be determined by limited trial and error, i.e. by introducing one or more envisaged humanizing substitutions and testing the humanized variants thus obtained for one or more desired properties, such as melting temperature, affinity, potency, properties upon formatting, expression levels in a desired host organism, and/or other desired properties for VHH domains or Nanobodies or proteins/polypeptides comprising the same, for which again reference is made to WO 09/068,627 and the further patent applications by applicant mentioned therein). For the purposes mentioned herein, it is not excluded that a humanizing substitution may also be introduced at a Camelid Hallmark residue, as long as this essentially does not detract (or does not detract too much) from the desired properties of the variant (in particular, the desired properties of VHH's and Nanobodies, as described in WO 09/068,627). Preferably, however, the humanizing substitutions are not at Camelid Hallmark residues (however, as described in the U.S. provisional application U.S. 61/358,495 by Ablynx N.V specifically for variants of 238D2).

Some particularly suitable variants of 238D2 that may be present in the amino acid sequences of the invention may for example be as described in the U.S. provisional application U.S. 61/358,495 by Ablynx N.V. filed on Jun. 25, 2010. As mentioned therein, such variants of 238D2 may be a variant of 238D2 (SEQ ID NO: 2) that comprises, compared to the amino acid sequence of 238D2, (i) at least one of the following mutations: T14P, M77T, Y82aN, K83R, and Q108L such as in immunoglobulin single variable domain of SEQ ID NO's: 23, 26 (ii) as well as optionally at least one, preferably at least two, and more preferably three, four of five humanizing substitutions; (iii) as well as optionally one or more further suitable amino acid substitutions. Thus, the invention provides an immunoglobulin single variable domain with any of SEQ ID NO's: 23 and 26.

Furthermore, for example, an epitope B binder may for example, and without limitation, be a variant of 238D4 (SEQ ID NO: 3) and comprise one or more (further) “humanizing” substitutions (as defined herein) and/or comprise one or more of the following substitutions, compared to the sequence of 238D4:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from WO 09/068,627, which mention the various Camelid residues that occur as each amino acid position in wild-type VHH's). Such substitutions may even comprise suitable substitutions of an amino acid residue that occurs at a Hallmark position with another amino acid residue that occurding at a Hallmark position in a wild-type VHH (for which reference is for example made to Tables A-6 to A-9 from WO 09/068,627); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific examples of suitable substitutions (see for example pages 4 and 5 and pages 10 to 15). One example of such substitution may be to replace an NS motif at positions 82a and 82b with an NN motif;
   or any suitable combination of two or more of any of the foregoing substitutions (a) to (c).

For the purposes described herein, a humanizing substitution can generally be defined as a substitution whereby an amino acid residue that occurs in a framework regions of a camelid V_{HH} domain is replaced by a different amino acid that occurs at the same position in the framework region of a human V_{H} domain (and preferably, a human V_{H}3 domain). Thus, suitable humanizing substitutions will be clear to the skilled person based on the disclosure herein, the disclosure in WO 09/068,627, and from a comparison of the amino acid sequence of a given V_{HH} sequence and one or more human V_{H} sequences.

Reference is for example made to the Tables A-6 to A-9 of WO 09/068,627, which list some of the amino acid residues that have been found to occur in the framework regions of camelid VHH domains, and the corresponding amino acid residue(s) that most often occur in the framework regions of a human V_{H}3 sequence (such as for example, the germline sequences DP-47, DP-51 or DP-29). The humanizing substitutions that can be taken from these Figures are also some of the preferred humanizing substitutions used in the invention; however, it may also be possible to use humanizing substitutions that have been obtained by comparison with other germline sequences (from the V_{H}3 class or sometimes also from other V_{H} classes). As generally known from WO 09/068,627 (and from the patent applications from Applicant and the further prior art mentioned in WO 09/068,627), based on such sequence comparison, particularly suited and/or optimal humanizing substitutions (and combinations thereof) may generally be determined by limited trial and error, i.e. by introducing one or more envisaged humanizing substitutions and testing the humanized variants thus obtained for one or more desired properties, such as melting temperature, affinity, potency, properties upon formatting, expression levels in a desired host organism, and/or other desired properties for VHH domains or Nanobodies or proteins/polypeptides comprising the same, for which again reference is made to WO 09/068,627 and the further patent applications by applicant mentioned therein). For the purposes mentioned herein, it is not excluded that a humanizing substitution may also be introduced at a Camelid Hallmark residue, as long as this essentially does not detract (or does not detract too much) from the desired properties of the variant (in particular, the desired properties of VHH's and Nanobodies, as described in WO 09/068,627). Preferably, however, the humanizing substitutions are not at Camelid Hallmark residues (however, as described in the U.S. provisional application U.S. 61/358,495 by Ablynx N.V specifically for variants of 238D4).

Some particularly suitable variants of 238D4 that may be present in the amino acid sequences of the invention may for example be as described in the U.S. provisional application U.S. 61/358,495 by Ablynx N.V. filed on Jun. 25, 2010. As mentioned therein, such variants of 238D4 may be a variant of 238D4 (SEQ ID NO: 4) that comprises, compared to the amino acid sequence of 238D4, (i) at least one of the following mutations: M5V, A14P, R39Q, K83R, T91Y, and Q108L such as in immunoglobulin single variable domain of SEQ ID NO's: 24, 25 (ii) as well as optionally at least one, preferably at least two, and more preferably three, four of five humanizing substitutions; (iii) as well as optionally one or more further suitable amino acid substitutions. Thus, the invention provides an immunoglobulin to single variable domain with any of SEQ ID NO's: 24 to 25.

### 3.) Polypeptides of the Invention

In a sixth aspect, the invention provides a polypeptide that is directed to and/or specifically binds to human CXCR4 and that at least comprises two or more immunoglobulin single variable domains of any of aspects one to five as described above in section 2).

In a seventh aspect, the invention provides a polypeptide of the sixth aspect, wherein the polypeptide comprises two immunoglobulin single variable domains.

In an eight aspect, the invention provides a polypeptide of the seventh aspect, wherein the immunoglobulin single variable domains are different.

In a ninth aspect, the invention provides a polypeptide of the seventh aspect, wherein the immunoglobulin single variable domains are different.

In a tenth aspect, the invention provides a polypeptide that is directed to and/or specifically binds to human CXCR4 and comprises:
a) an immunoglobulin single variable domain that can specifically bind to an epitope A that comprises and/or essentially consists of the conformation dependent epitope with the amino acid residues F189, N192, W195, P191, V196 and optionally E277, wherein the numbering of the amino acid residues refers to the human CXCR4-short sequence (SEQ ID NO: 6); and
b) an immunoglobulin single variable domain that can specifically bind to an epitope B that comprises and/or essentially consists of the conformation dependent epitope with the amino acid residues D187, F189, E179 and S178, wherein the numbering of the amino acid residues refers to the human CXCR4-short sequence (SEQ ID NO: 6); and
c) wherein neither of the above immunoglobulin single variable domains is an amino acid sequence selected from the group consisting of 238D2 (SEQ ID NO: 2) or 238D4 (SEQ ID NO: 3); and
wherein optionally the polypeptide has i) a maximal displacement of human CXCL12 (SDF-1) on human CXCR4 expressing HEK293T cells by more than 90% and ii) a receptor affinity Ki of 1 nM or lower to said human CXCR4 expressing HEK293T cells (as e.g. measured in Example 4.2 of WO2009/138519).

In an eleventh aspect, the invention provides a polypeptide comprising any of the above immunoglobulin single variable domains that are linked with a peptide selected from to the group of peptides consisting of amino acid sequences with SEQ ID NOs: 7 to 16 (Table B-3).

In a twelfth aspect, the invention provides a polypeptide that is directed to and/or specifically binds to human CXCR4 and comprises:
a) a first (i.e. N-terminal of the second) immunoglobulin single variable domain that specifically binds to an epitope A that comprises and/or essentially consists of the conformation dependent epitope with the amino acid residues F189, N192, W195, P191, V196 and optionally E277, wherein the numbering of the amino acid residues refers to the human CXCR4-short sequence (SEQ ID NO: 6); and
b) a second (i.e. C-terminal of the first, optionally linked by a peptide) immunoglobulin single variable domain that can specifically bind to an epitope B that comprises and/or essentially consists of the conformation dependent epitope with the amino acid residues D187, F189, E179 and S178, wherein the numbering of the amino acid residues refers to the human CXCR4-short sequence (SEQ ID NO: 6); and
c) wherein neither of the above immunoglobulin single variable domains is an amino acid sequence selected from the group consisting of 238D2 (SEQ ID NO: 2) or 238D4 (SEQ ID NO: 3); and
d) wherein optionally the two immunoglobulin single variable domains are linked by a peptide that is e.g. selected from the group of peptides consisting of amino acid sequences with SEQ ID NOs: 7 to 16 (Table B-3), preferably SEQ ID NO: 13; and
   wherein optionally the polypeptide has i) a maximal displacement of human CXCL12 (SDF-1) on human CXCR4 expressing HEK293T cells by more than 90% and ii) a receptor affinity Ki of 1 nM or lower to said human CXCR4 expressing HEK293T cells (as e.g. measured in Example 4.2 of WO2009/138519).

These polypeptides may optionally further contain one or more suitable linkers, spacers, and/or other amino acid sequences, moieties, residues, binding domains, binding units or binding sites, as for example described in WO2009/138519 and may be in particular half life-extending moieties as described in the below examples.

Some non-limiting examples of such polypeptides comprising epitope A and/or epitope B binders may be represented as follows (with the N-terminus of the polypeptide towards the right and the C-terminus towards the left):
[epitope A binder]-linker-[epitope B binder],
[epitope A binder]-linker-[non-epitope B binder],
[non-epitope A binder]-linker-[epitope B binder],
[epitope A binder]-linker-[epitope B binder], which construct may optionally be pegylated for increased half-life in circulation;
[epitope A binder]-linker-[Nanobody/VHH binding to serum albumin, such as Alb-11 (SEQ ID NO: 17)]-linker-[epitope B binder];
[epitope A binder]-linker-[Nanobody/VHH binding to serum albumin, such as Alb-11 (SEQ ID NO: 17)]-linker-[non-epitope B binder];
[non-epitope A binder]-linker-[Nanobody/VHH binding to serum albumin, such as Alb-11 (SEQ ID NO: 17)]-linker-[epitope B binder];
[epitope A binder]-linker-[epitope B binder]-linker-[Nanobody/VHH binding to serum albumin, such as Alb-11 (SEQ ID NO: 17)];
[epitope A binder]-linker-[non-epitope B binder]-linker-[Nanobody/VHH binding to serum albumin, such as Alb-11 (SEQ ID NO: 17)];
[non-epitope A binder]-linker-[epitope B binder]-linker-[Nanobody/VHH binding to serum albumin, such as Alb-11 (SEQ ID NO: 17)];
[serum albumin]-linker-[epitope A binder]-linker-[epitope B binder];
[epitope A binder]-linker-[epitope B binder]-linker-[serum albumin]
[serum albumin binding peptide (monovalent or in tandem)]-[epitope A binder]-linker-[epitope B binder];
[serum albumin binding peptide (monovalent or in tandem)]-[epitope A binder]-linker-[non-epitope B binder];
[serum albumin binding peptide (monovalent or in tandem)]-[non-epitope A binder]-linker-[epitope B binder];
[epitope A binder]-linker-[epitope B binder]-[serum albumin binding peptide (monovalent or in tandem)];
[epitope A binder]-linker-[non-epitope B binder]-[serum albumin binding peptide (monovalent or in tandem)];
[non-epitope A binder]-linker-[epitope B binder]-[serum albumin binding peptide (monovalent or in tandem)].

For the serum albumin binding peptide, reference is e.g. made to WO 2009/127691. The above polypeptides and/or immunoglobulin single variable domains may optionally be tagged with tags known to the skilled person such as e.g. 3×Flag-His6 (SEQ ID NO: 18).

Some particularly suitable variants of 238D2-20GS-238D4 that may be present in the amino acid sequences of the invention may for example be as described in the U.S. provisional application U.S. 61/358,495 by Ablynx N.V. filed on Jun. 25, 2010. As mentioned therein, such variants may be a variant that comprises, compared to the amino acid sequence of 238D2-20GS-238D4, (i) at least one of the following mutations in the 238D2 building block: any or all of T14P, M77T, Y82aN, K83R, and Q108L (using the well known Kabat numbering system) and in the 238D4 building block: any or all of M5V, A14P, R39Q, K83R, T91Y, and Q108L (using the well known Kabat numbering system) such as in the polypeptides of SEQ ID NO's: 27 to 30 (ii) as well as optionally at least one, preferably at least two, and more preferably three, four of five humanizing substitutions; (iii) as well as optionally one or more further suitable amino acid substitutions. Thus, the invention provides polypeptides of any of SEQ ID NO's: 27 to 30.

### 4.) Methods to Generate the Compounds and Other Related Materials of the Invention Using the Particular Epitopes Identified on Human CXCR4

The invention further relates to methods for preparing or generating the immunoglobulin single variable domains, polypeptides, nucleic acids, host cells, products and compositions described herein. Some preferred but non-limiting examples of such methods will become clear from the further description herein.

Generally, these methods may comprise the steps of:
a) providing a set, collection or library of immunoglobulin single variable domains; and
b) screening said set, collection or library of immunoglobulin single variable domains for immunoglobulin single variable domains that can bind to and/or have affinity for CXCR4 and in particular human CXCR4; and
c) isolating the immunoglobulin single variable domains that can bind to and/or have affinity for CXCR4 and in particular human CXCR4; and
d) and selecting an isolated amino acid sequence(s) from the group of amino acid sequences from c) that i) displaces to 90%, more preferably 95%, most preferred 99% 238D2 (SEQ ID NO: 2) and/or 238D4 (SEQ ID NO: 3) in a displacement assay such as e.g. shown in the experimental part; and ii) has a binding pattern in e.g. the footprint assay of the invention (see experimental part) of an epitope A- or epitope B binder.

In such a method, the set, collection or library of immunoglobulin single variable domains may be any suitable set, collection or library of immunoglobulin single variable domains. For example, the set, collection or library of immunoglobulin single variable domains may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of immunoglobulin single variable domains may be a set, collection or library of heavy or light chain variable domains (such as VL-, VH- or VHH domains, preferably VHH domains). For example, the set, collection or library of immunoglobulin single variable domains may be a set, collection or library of domain antibodies or single domain antibodies, or may be a set, collection or library of immunoglobulin single variable domains that are capable of functioning as a domain antibody or single domain antibody.

In a preferred aspect of this method, the set, collection or library of immunoglobulin single variable domains may be an immune set, collection or library of immunoglobulin sequences, for example derived from a mammal that has been suitably immunized with CXCR4 and in particular human CXCR4 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of immunoglobulin single variable domains may be displayed on a phage, phagemid, ribosome or suitable micro-organism (such as yeast), such as to facilitate screening. Suitable methods, techniques and host organisms for displaying and screening (a set, collection or library of) immunoglobulin single variable domains will be clear to the person skilled in the art, for example on the basis of the further disclosure herein. Reference is also made to the review by Hoogenboom in Nature Biotechnology, 23, 9, 1105-1116 (2005).

In another aspect, the method for generating immunoglobulin single variable domains comprises at least the steps of:
a) providing a collection or sample of cells expressing immunoglobulin single variable domains; and
b) screening said collection or sample of cells for cells that express an amino acid sequence that can bind to and/or have affinity for CXCR4 and in particular human CXCR4; and
c) either (i) isolating said amino acid sequence; or (ii) isolating from said cell a nucleic acid sequence that encodes said amino acid sequence, followed by expressing said amino acid sequence; and
d) and selecting an isolated amino acid sequence(s) from the group of amino acid sequences from c) that i) displaces to 90%, more preferably 95%, most preferred 99% 238D2 (SEQ ID NO: 2) and/or 238D4 (SEQ ID NO: 3) in a displacement assay such as e.g. shown in the experimental part; and ii) has a binding pattern in e.g. the footprint assay of the invention (see experimental part) of an epitope A- or epitope B binder.

In another aspect, the method for generating an amino acid sequence directed against CXCR4 and in particular human CXCR4 may comprise at least the steps of:
a) providing a set, collection or library of nucleic acid sequences encoding immunoglobulin single variable domains; and
b) screening said set, collection or library of nucleic acid sequences for nucleic acid sequences that encode an amino acid sequence that can bind to and/or has affinity for CXCR4 and in particular human CXCR4; and
c) isolating said nucleic acid sequence, followed by expressing said amino acid sequence; and
d) and selecting an isolated amino acid sequence(s) from the group of amino acid sequences from c) that i) displaces to 90%, more preferably 95%, most preferred 99% 238D2 (SEQ ID NO: 2) and/or 238D4 (SEQ ID NO: 3) in a displacement assay such as e.g. shown in the experimental part; and ii) has a binding pattern in e.g. the footprint assay of the invention (see experimental part) of an epitope A- or epitope B binder.

In such a method, the set, collection or library of nucleic acid sequences encoding immunoglobulin single variable domains may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

In another aspect, the method for generating an amino acid sequence directed against CXCR4 and in particular human CXCR4 may comprise at least the steps of:
a) providing a set, collection or library of nucleic acid sequences encoding immunoglobulin single variable domains; and
b) screening said set, collection or library of nucleic acid sequences for nucleic acid sequences that encode an amino acid sequence that can bind to and/or has affinity for CXCR4 and in particular human CXCR4 and that is cross-blocked or is cross blocking a reference immunoglobulin single variable domain or polypeptide, e.g. a compound selected from the group consisting of SEQ ID NO's: 2 to 5; and
c) isolating said nucleic acid sequence, followed by expressing said amino acid sequence; and
d) and selecting an isolated amino acid sequence(s) from the group of amino acid sequences from c) that i) displaces to 90%, more preferably 95%, most preferred 99% 238D2 (SEQ ID NO: 2) and/or 238D4 (SEQ ID NO: 3) in a displacement assay such as e.g. shown in the experimental part; and ii) has a binding pattern in e.g. the footprint assay of the invention (see experimental part) of an epitope A- or epitope B binder.

The invention also relates to immunoglobulin single variable domains that are obtained by the above methods, or alternatively by a method that comprises the one of the above methods and in addition at least the steps of determining the nucleotide sequence or amino acid sequence of said immunoglobulin sequence; and of expressing or synthesizing said amino acid sequence in a manner known per se, such as by expression in a suitable host cell or host organism or by chemical synthesis.

Also, following the steps above, one or more immunoglobulin single variable domains of the invention may be suitably humanized, camilized or otherwise sequence optimized (e.g. sequence optimized for manufacturablity, stability and/or solubility); and/or the amino acid sequence(s) thus obtained may be linked to each other or to one or more other suitable immunoglobulin single variable domains (optionally via one or more suitable linkers) so as to provide a polypeptide of the invention. Also, a nucleic acid sequence encoding an amino acid sequence of the invention may be suitably humanized, camilized or otherwise sequence optimized (e.g. sequence optimized for manufacturablity, stability and/or solubility) and suitably expressed; and/or one or more nucleic acid sequences encoding an amino acid sequence of the invention may be linked to each other or to one or more nucleic acid sequences that encode other suitable immunoglobulin single variable domains (optionally via nucleotide sequences that encode one or more suitable linkers), after which the nucleotide sequence thus obtained may be suitably expressed so as to provide a polypeptide of the invention.

5.) Pharmaceutical Composition Comprising the Compounds of the Invention and Uses Thereof in the Treatment, Diagnosis and/or Prevention of Diseases and/or Disorders

Generally, for pharmaceutical use, the polypeptides of the invention may be formulated as a pharmaceutical preparation or composition comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. wherein which the parenteral administration is preferred. Such suitable administration forms—which may be solid, semi-solid or liquid, depending on the manner of administration—as well as methods and carriers for use in the preparation thereof, will be clear to the skilled person, and are further described herein. Such a pharmaceutical preparation or composition will generally be referred to herein as a “pharmaceutical composition”. A pharmaceutical preparation or composition for use in a non-human organism will generally be referred to herein as a “veterinary composition”.

Thus, in a further aspect, the invention relates to a pharmaceutical composition that contains at least one amino acid of the invention, at least one polypeptide of the invention or at least one polypeptide of the invention and at least one suitable carrier, diluent or excipient (i.e., suitable for pharmaceutical use), and optionally one or more further active substances.

Generally, the polypeptides of the invention can be formulated and administered in any suitable manner known per se. Reference is for example made to the general background art cited above (and in particular to WO 04/041862, WO 04/041863, WO 04/041865, WO 04/041867 and WO 08/020,079) as well as to the standard handbooks, such as Remington's Pharmaceutical Sciences, 18^{th}., Ea Mack Publishing Company, USA (1990), Remington, the Science and Practice of Pharmacy, 21th Edition, Lippincott Williams and Wilkins (2005); or the Handbook of Therapeutic Antibodies (S. Dubel, Ed.), Wiley, Weinheim, 2007 (see for example pages 252-255).

The polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e., transdermal or intradermal) administration.

Preparations for parenteral administration may for example be sterile solutions, suspensions, dispersions or emulsions that are suitable for infusion or injection. Suitable carriers or diluents for such preparations for example include, without limitation, those mentioned on page 143 of WO 08/020,079. In one embodiment, the preparation is an aqueous solution or suspension.

The polypeptides of the invention can be administered using gene therapy methods of delivery. See, e.g., U.S. Pat. No. 5,399,346, which is incorporated by reference for its gene therapy delivery Methods. Using a gene therapy method of delivery, primary cells transfected with the gene encoding an amino acid sequence, polypeptide of the invention can additionally be transfected with tissue specific promoters to target specific organs, tissue, grafts, tumors, or cells and can additionally be transfected with signal and stabilization sequences for subcellularly localized expression.

Thus, the polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the polypeptides of the invention may be combined with one or more excipients and used in the form of ingestible tablets, buccal tablets, troches, capsules, elixirs, suspensions, syrups, wafers, and the like. Such compositions and to preparations should contain at least 0.1% of the polypeptide of the invention. Their percentage in the compositions and preparations may, of course, be varied and may conveniently be between about 2 to about 60% of the weight of a given unit dosage form. The amount of the polypeptide of the invention in such therapeutically useful compositions is such that an effective dosage level will be obtained.

For local administration at the site of tumor resection, the polypeptides of the invention may be used in biodegradable polymeric drug delivery systems, slow release poly(lactic-co-glycolic acid formulations and the like (Hart et al., Cochrane Database Syst Rev. 2008 Jul. 16; (3): CD007294).

In a further preferred aspect of the invention, the immunoglobulin single variable domains and/or polypeptides of the invention may have a beneficial distribution and kinetics profile in solid tumors compared to conventional antibodies such as e.g. IgG.

The tablets, troches, pills, capsules, and the like may also contain binders, excipients, disintegrating agents, lubricants and sweetening or flavoring agents, for example those mentioned on pages 143-144 of WO 08/020,079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the polypeptides of the invention, sucrose or fructose as a sweetening agent, Methyl and propylparabens as preservatives, a dye and flavoring such as cherry or orange flavor. Of course, any material used in preparing any unit dosage form should be pharmaceutically acceptable and substantially non-toxic in the amounts employed. In addition, the polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The polypeptides of the invention may also be administered intravenously or to intraperitoneally by infusion or injection. Particular examples are as further described on pages 144 and 145 of WO 08/020,079, PCT/EP2010/062975 (entire document).

For topical administration, the polypeptides of the invention may be applied in pure form, i.e., when they are liquids. However, it will generally be desirable to administer them to the skin as compositions or formulations, in combination with a dermatologically acceptable carrier, which may be a solid or a liquid. Particular examples are as further described on page 145 of WO 08/020,079.

Generally, the concentration of the polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the polypeptides of the invention required for use in treatment will vary not only with the particular polypeptide selected but also with the route of administration, the nature of the condition being treated and the age and condition of the patient and will be ultimately at the discretion of the attendant physician or clinician. Also the dosage of the polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

In another aspect, the invention relates to a method for the prevention and/or treatment of at least one diseases and disorders associated with CXCR4, said method comprising administering, to a subject in need thereof, a pharmaceutically active amount of a polypeptide of the invention, and/or of a pharmaceutical composition comprising the same. The invention further relates to applications and uses of the immunoglobulin single variable domains, to compounds, constructs, polypeptides, nucleic acids, host cells, products and compositions described herein, as well as to methods for the diagnosis, prevention and/or treatment for diseases and disorders associated with CXCR4 and in particular human CXCR4. Some preferred but non-limiting applications and uses will become clear from the further description herein.

The invention also relates to the immunoglobulin single variable domains, compounds, constructs, polypeptides, nucleic acids, host cells, products and compositions described herein for use in therapy.

In particular, the invention also relates to the immunoglobulin single variable domains, compounds, constructs, polypeptides, nucleic acids, host cells, products and compositions described herein for use in therapy of a disease or disorder that can be prevented or treated by administering, to a subject in need thereof, of (a pharmaceutically effective amount of) an amino acid sequence, compound, construct or polypeptide as described herein.

More in particular, the invention relates to the immunoglobulin single variable domains, compounds, constructs, polypeptides, nucleic acids, host cells, products and compositions described herein for use in therapy of cancer.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention relates to a method for the prevention and/or treatment of at least one disease or disorder that is associated with CXCR4, with its biological or pharmacological activity, and/or with the biological pathways or signaling in which CXCR4 is involved, said method comprising administering, to a subject in need thereof, a pharmaceutically active amount of an amino acid sequence of the invention, of a polypeptide of the invention, of a polypeptide of the invention, and/or of a pharmaceutical composition comprising the same. In one embodiment, the invention relates to a method for the prevention and/or treatment of at least one disease or disorder that can be treated by modulating CXCR4, its biological or pharmacological activity, and/or the biological pathways or signaling in which CXCR4 are involved, said method comprising administering, to a subject in need thereof, a pharmaceutically active amount of a polypeptide of the invention, and/or of a pharmaceutical composition comprising the same. In one embodiment, said pharmaceutically effective amount may be an amount that is sufficient to modulate CXCR4, its biological or pharmacological activity, and/or the biological pathways or signaling in which CXCR4 is involved; and/or an amount that provides a level of the polypeptide of the invention in the circulation that is sufficient to modulate CXCR4, its biological or pharmacological activity, and/or the biological pathways or signaling in which CXCR4 is involved.

In one embodiment the invention relates to a method for the prevention and/or treatment of at least one disease or disorder that can be prevented and/or treated by administering a polypeptide of the invention, or a nucleotide construct of the invention encoding the same, and/or of a pharmaceutical composition comprising the same, to a patient. In one embodiment, the method comprises administering a pharmaceutically active amount of a polypeptide of the invention, or a nucleotide construct of the invention encoding the same, and/or of a pharmaceutical composition comprising the same to a subject in need thereof.

In one embodiment the invention relates to a method for the prevention and/or treatment of at least one disease or disorder that can be prevented and/or treated by inhibiting binding of SDF-1a to CXCR4 in specific cells or in a specific tissue of a subject to be treated (and in particular, by inhibiting binding of SDF-1a to CXCR4 in cancer cells or in a tumor present in the subject to be treated), said method comprising administering a pharmaceutically active amount of a polypeptide of the invention, or a nucleotide construct of the invention encoding the same, and/or of a pharmaceutical composition comprising the same, to a subject in need thereof.

In one embodiment, the invention relates to a method for the prevention and/or treatment of at least one disease or disorder chosen from the group consisting of the diseases and disorders listed herein, said method comprising administering, to a subject in need thereof, a polypeptide of the invention, or a nucleotide construct of the invention encoding the same, and/or of a pharmaceutical composition comprising the same.

In one embodiment, the invention relates to a method for immunotherapy, and in particular for passive immunotherapy, which method comprises administering, to a subject suffering from or at risk of the diseases and disorders mentioned herein, a pharmaceutically active amount of a polypeptide of the invention, or a nucleotide construct of the invention encoding the same, and/or of a pharmaceutical composition comprising the same.

In the above methods, the amino acid sequences, polypeptides of the invention and/or the compositions comprising the same can be administered in any suitable manner, depending on the specific pharmaceutical formulation or composition to be used. Thus, the polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more polypeptides of the invention, or of one or more compositions comprising the same, in one or more pharmaceutically effective amounts or doses. The specific amount(s) or doses to be administered can be determined by the clinician, again based on the factors cited above.

Generally, for the prevention and/or treatment of the diseases and disorders mentioned herein and depending on the specific disease or disorder to be treated, the potency of the specific polypeptide of the invention to be used, the specific route of administration and the specific pharmaceutical formulation or composition used, the polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

The polypeptides or compounds of the invention can be used for the same purposes, uses and applications as described in WO 09/138,519, for example to inhibit signaling that is mediated by human CXCR4 and/or its ligand(s); and/or in the prevention or treatment of diseases associated with an increased signalling of CXCR4, such as the various diseases in the group of cancer such as hematopoietic cancers like CLL, AML, ALL, MM, Non-Hodgkin lymphoma, solid tumors such as breast cancer, lung cancer, brain tumors, ovarian cancer, stromal chemoresistance of tumors, leukemia and other cancers, disrupting adhesive stromal interactions that confer tumor cell survival and drug resistance, mobilizing tumor cells form tissue sites and making them better accessible to conventional therapy, inhibiting of migration and dissemination of tumor cells (metastasis), inhibiting or paracrine growth and survival signals, inhibiting pro-angiogenesis effects of SDF-1, inflammation and inflammatory disorders such as bowel diseases (colitis, Crohn's disease, IBD), infectious diseases, psioriasis, autoimmune diseases (such as MS), sarcoidosis, transplant rejection, cystic fibrosis, asthma, chronic obstructive pulmonary disease, rheumatoid arthritis, viral infection, HIV, West Nile Virus encephalitis, common variable immunodeficiency. Furthermore, the amino acid sequences of the invention can be used for stem cell mobilization in various patients in need of stem cells after X-ray radiation such as e.g. cancer patients after radiation treatment to replenish the stem cell pool after radiation in cancer patients, or in patients in need of more stem cells, e.g. in patients with ischemic diseases such as myocardial infarction (MI), stroke and/or diabetes (i.e. patients in need of tissue repair) wherein more stem cell would be re-transfused (after mobilization, screening, selection for lineage in need (e.g. cardiac, vascular lineages) and ex-vivo expansion of patient's own stem cells).

In particular, the polypeptides and compounds of the invention are very potent (i.e. EC50 values as measured e.g. in the experimental part in the sub nM range) antagonists of human CXCR4 and/or are inverse agonists in certain continuously active human CXCR4 mutants (see e.g. example 5 of WO 09/138,519). Reference is for example made to examples 5 and 6 on pages 222ff of WO 09/138,519, as well as the further general disclosure of WO 09/138,519. More in particular, the polypeptides of the invention may be used as an improved alternative to 238D2-20GS-238D4, and thus may in particular be used for the same purposes as described in WO 09/138,519 for 238D2-20GS-238D4.

In one embodiment, a single contiguous polypeptide of the invention will be used. In one embodiment two or more polypeptides of the invention are provided in combination.

The polypeptides of the invention may be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgment.

In particular, the polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician, and generally include the cytostatic active principles usually applied for the treatment of the tumor to be treated.

Specific contemplated combinations for use with the polypeptides of the invention for oncology include, but are not limited to, e.g., RON antagonists, chemokine receptor antagonists, taxol; gemcitobine; cisplatin; cIAP inhibitors (such as inhibitors to cIAP1, cIAP2 and/or XIAP); MEK inhibitors including but not limited to, e.g., U0126, PD0325901; bRaf inhibitors including but not limited to, e.g., RAF265; and mTOR inhibitors including but not limited to, e.g., RAD001; VEGF inhibitors including but not limited to e.g. bevacizumab, sutinib and sorafenib; Her2 inhibitors including but not limited to e.g. trastuzumab and lapatinib; EGFR, Her3, Her4, PDGFR, FGFR, src, JAK, STAT and/or GSK3 inhibitors; selective estrogen receptor modulators including but not limited to tamoxifen; estrogen receptor downregulators including but not limited to fulvestrant. Specific contemplated combinations for use with the polypeptides of the invention for inflammatory conditions include, but are not limited to, e.g., interferon beta 1 alpha and beta, natalizumab; TNF alpha antagonists including but not limited to e.g. infliximab, adalimumab, certolizumab pegol, etanercept; disease-modifying antirheumatic drugs such as e.g. Methotrexate (MTX); glucocortioids including but not limited to e.g. hydrocortisone; Nonsteroidal anti-inflammatory drugs including but not limited to e.g. ibuprofen, sulindac.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of polypeptide of the invention in the preparation of a pharmaceutical composition for prevention and/or treatment of at least one disease and disorder associated with CXCR4; and/or for use in one or more of the methods of treatment mentioned herein.

The subject to be treated may be any warm-blooded animal, but is in particular a mammal, and more in particular a human being. In veterinary applications, the subject to be treated includes any animal raised for commercial purposes or kept as a pet. As will be clear to the skilled person, the subject to be treated will in particular be a person suffering from, or at risk of, the diseases and disorders mentioned herein.

The invention relates to the use of a polypeptide of the invention, or a nucleotide encoding the same, in the preparation of a pharmaceutical composition for the prevention and/or treatment of at least one disease or disorder that can be prevented and/or treated by administering a polypeptide of the invention, or a nucleotide encoding the same, and/or a pharmaceutical composition of the same to a patient.

More in particular, the invention relates to the use of a polypeptide of the invention, or a nucleotide encoding the same, in the preparation of a pharmaceutical composition for the prevention and/or treatment of diseases and disorders associated with CXCR4, and in particular for the prevention and treatment of one or more of the diseases and disorders listed herein.

Again, in such a pharmaceutical composition, the one or more polypeptide of the invention, or nucleotide encoding the same, and/or a pharmaceutical composition of the same, may also be suitably combined with one or more other active principles, such as those mentioned herein.

The invention also relates to a composition (such as, without limitation, a pharmaceutical composition or preparation as further described herein) for use, either in vitro (e.g. in an in vitro or cellular assay) or in vivo (e.g. in an a single cell or multicellular organism, and in particular in a mammal, and more in particular in a human being, such as in a human being that is at risk of or suffers from a disease or disorder of the invention).

In the context of the present invention, “modulating” or “to modulate” generally means reducing or inhibiting the activity of CXCR4 and in particular human CXCR4, as measured using a suitable in vitro, cellular or in vivo assay (such as those mentioned herein). In particular, reducing or inhibiting the activity of CXCR4 and in particular human CXCR4, as measured using a suitable in vitro, cellular or in vivo assay (such as those mentioned herein), by at least 1%, preferably at least 5%, such as at least 10% or at least 25%, for example by at least 50%, at least 60%, at least 70%, at least 80%, or 90% or more, compared to activity of CXCR4 and in particular human CXCR4 in the same assay under the same conditions but without the presence of the polypeptide of the invention.

Modulating may for example involve reducing or inhibiting the binding CXCR4 to one of its substrates or ligands and/or competing with natural ligands (HGF), substrate for binding to CXCR4. Alternatively, modulating may involve inhibiting the internalization, homodimerization of CXCR4 and/or promoting of shedding of CXCR4 and thus may inhibit HGF dependent and/or HGF independent CXCR4 activation.

6.) Method to Generate Other (than the Above Described) Compounds of the Invention Using Epitope Walking with Multimeric Libraries Methodology

The present invention also relates to a method for the generation and/or identification of an immunoglobulin single variable domain that can bind to and/or has affinity for an epitope of a cell-associated antigen; wherein said immunoglobulin single variable domain is not cross-blocked or only partly cross-blocked by the first immunoglobulin single variable domain (or a polypeptide comprising said first immunoglobulin single variable domain, e.g. 2 of said first immunoglobulin single variable domains), comprising the steps of:
a) generation of a set, collection or library of fusion proteins, wherein said fusion protein is displayed on e.g. a virus such as a phage and wherein said fusion protein comprises
   a. a first immunoglobulin single variable domain (or a polypeptide comprising said first immunoglobulin single variable domain, e.g. 2 of said first immunoglobulin single variable domains) that is known to bind to said cell-associated antigen; and
   b. a linker; and
   c. a second immunoglobulin single variable domain selected from a set, collection or library of immunoglobulin single variable domains; wherein said set, collection or library of immunoglobulin single variable domains is optionally depleted of dominant binders during the cloning procedure;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning, epitope analysis, ligand competition assay, and/or functional assays;
e) and optionally producing said generated and/or identified second immunoglobulin single variable domain (or a polypeptide comprising said second immunoglobulin single variable domain),
f) and optionally repeating steps above until an immunoglobulin single variable domain (or a polypeptide comprising said second immunoglobulin single variable domain) is obtained with a non overlapping or partially overlapping epitope.

Thus, in general terms the method of the present invention includes generation and/or identification of an immunoglobulin single variable domain as defined herein. In one particular embodiment, the immunoglobulin single variable domain is a Nanobody. Thus, in a specific embodiment, the method for the generation and/or identification of a Nanobody that can bind to and/or has affinity for an epitope; wherein said immunoglobulin single variable domain is not cross-blocked or only partly cross-blocked by the first immunoglobulin single variable domain (or a polypeptide comprising said first immunoglobulin single variable domain, e.g. 2 of said first immunoglobulin single variable domains), of a cell-associated antigen comprising the steps of:
a) generation of a set, collection or library of fusion proteins, wherein said fusion protein is displayed on e.g. a virus such as a phage, preferably on a phage (if displayed on a phage, the set, collection or library is also referred herein as “Nanobody-fusion phage library”) and wherein said fusion protein comprises
   a. a first Nanobody sequence (or a polypeptide comprising said first Nanobody sequence, e.g. multimeric Nanobody) that is known to bind to said cell-associated antigen; and
   b. a linker such as e.g. 5 GS to 40 GS; and
   c. a second Nanobody sequence selected from a set, collection or library of immunoglobulin single variable domains; wherein said set, collection or library of immunoglobulin single variable domains is optionally depleted of dominant binders during the cloning procedure;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning (e.g. blocking of non-functional and/or unwanted dominant epitopes by available monoclonal antibodies and/or other Nanobodies), epitope analysis, ligand competition assay, and/or functional assays;
e) and optionally producing said generated and/or identified second Nanobody (or a polypeptide comprising said second Nanobody),
f) and optionally repeating steps above until a Nanobody is obtained with e.g. a different function or wherein a polypeptide comprising said second Nanobody or further Nanobody obtains a new function (e.g. shift from antagonist to inverse agonist).

A particular advantage of the present invention resides in the fact that it provides a method for generating immunoglobulin single variable domains, such as e.g. Nanobodies, to an epitope that is normally not accessible by standard methods. Once a first binding immunoglobulin single variable domain is identified by e.g. standard methods, the method can then e.g. be used to obtain a second (and third or further) immunoglobulin single variable domain that recognises a different epitope, and said second (or third or further) binder either alone or fused to said first and/or second binder is functional, e.g. has an agonistic, antagonistic or inverse agonistic effect. The method exploits the fact that by using this method the local antigen concentration for the second (and third) binding interaction and/or the avidity effect of the immunoglobulin single variable domain is increased. Moreover, non functional and/or dominant epitopes can be further “blended” out a) by masking said epitopes by the first (and/or second) immunoglobulin single variable domain known to bind to the antigen and/or b) by depleting known dominant binders from the set, collection or library of immunoglobulin single variable domains during the cloning procedure (see examples). The method of the invention is not limited by the difficult (=not accessible by standard methods) accessibility of protein antigen. In particular, there is no requirement for purified antigen. Hence, the method of the present invention is broadly applicable to any of the antigens exemplified above, but not limited thereto.

Hence, the present invention is advantageous as compared to prior art methods that lack such applicability. In particular there is no teaching in the art for such a method for the generation of immunoglobulin single variable domains in animals such as camelids, in particular llama.

Specifically, the present invention provides an improved method for generating immunoglobulin single variable domains against cell-associated antigens, which, according to one specific embodiment, is in particular suitable for the generation of Nanobodies to particular epitopes of choice.

More particularly, the present invention provides a method for the generation of immunoglobulin single variable domains, including Nanobodies, against an epitope of a cell-associated antigen that is a modulator of said cell-associated antigen, comprising the steps of:
a) generation of a set, collection or library of fusion proteins, wherein said fusion protein is displayed on e.g. a virus such as a phage and wherein said fusion protein comprises
   a. first immunoglobulin single variable domain (or a polypeptide comprising said first immunoglobulin single variable domain, e.g. 2 of said first immunoglobulin single variable domains) that is known to bind to said cell-associated antigen; and
   b. a linker; and
   c. a second immunoglobulin single variable domain selected from a set, collection or library of immunoglobulin single variable domains;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning, epitope analysis, ligand competition assay, and/or functional assays;
e) and optionally producing said generated and/or identified second immunoglobulin single variable domain (or a polypeptide comprising said second immunoglobulin single variable domain),
f) and repeating steps above until an immunoglobulin single variable domain (or a polypeptide comprising said second immunoglobulin single variable domain) is obtained that is a modulator of said cell-associated antigen.

More particularly, the present invention provides a method for the generation of immunoglobulin single variable domains, including Nanobodies, against an epitope of a cell-associated antigen that is an antagonist of said cell-associated antigen, comprising the steps of:
a) generation of a set, collection or library of fusion proteins, wherein said fusion protein is displayed on e.g. a virus such as a phage and wherein said fusion protein comprises
   a. first immunoglobulin single variable domain (or a polypeptide comprising said first immunoglobulin single variable domain, e.g. 2 of said first immunoglobulin single variable domains) that is known to bind to said cell-associated antigen; and
   b. a linker; and
   c. a second immunoglobulin single variable domain selected from a set, collection or library of immunoglobulin single variable domains;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning, epitope analysis, ligand competition assay, and/or functional assays;
e) and optionally producing said generated and/or identified second immunoglobulin single variable domain (or a polypeptide comprising said second immunoglobulin single variable domain),
f) and repeating steps above until an immunoglobulin single variable domain (or a polypeptide comprising said second immunoglobulin single variable domain) is obtained that is an antagonist of said cell-associated antigen.

More particularly, the present invention provides a method for the generation of immunoglobulin single variable domains, including Nanobodies, against an epitope of a cell-associated antigen that is an agonist of said cell-associated antigen, comprising the steps of:
a) generation of a set, collection or library of fusion proteins, wherein said fusion protein is displayed on e.g. a virus such as a phage and wherein said fusion protein comprises
   a. first immunoglobulin single variable domain (or a polypeptide comprising said first immunoglobulin single variable domain, e.g. 2 of said first immunoglobulin single variable domains) that is known to bind to said cell-associated antigen; and
   b. a linker; and
   c. a second immunoglobulin single variable domain selected from a set, collection or library of immunoglobulin single variable domains;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning, epitope analysis, ligand competition assay, and/or functional assays;
e) and optionally producing said generated and/or identified second immunoglobulin single variable domain (or a polypeptide comprising said second immunoglobulin single variable domain),
f) and repeating steps above until an immunoglobulin single variable domain (or a polypeptide comprising said second immunoglobulin single variable domain) is obtained that is an agonist of said cell-associated antigen.

More particularly, the present invention provides a method for the generation of immunoglobulin single variable domains, including Nanobodies, against an epitope of a cell-associated antigen that is an inverse agonist of said cell-associated antigen, comprising the steps of:
a) generation of a set, collection or library of fusion proteins, wherein said fusion protein is displayed on e.g. a virus such as a phage and wherein said fusion protein comprises
   a. first immunoglobulin single variable domain (or a polypeptide comprising said first immunoglobulin single variable domain, e.g. 2 of said first immunoglobulin single variable domains) that is known to bind to said cell-associated antigen; and
   b. a linker; and
   c. a second immunoglobulin single variable domain selected from a primary set, collection or library of immunoglobulin single variable domains;
b) and selection of said displayed set, collection or library of fusion proteins against said cell-associated antigen in its natural conformation, e.g. wherein said cell-associated antigen is selected from cells comprising natural or transfected cells expressing the cell-associated antigen, cell derived membrane extracts, vesicles or any other membrane derivative harbouring enriched antigen, liposomes, lipoprotein particles, nanolipoprotein particles or virus particles expressing said cell-associated antigen;
c) and optionally further selecting fusion proteins or its nucleotide sequence respectively from step b) via PCR for nucleotide sequences encoding for fusion proteins comprising an immunoglobulin single variable domain that is/are different from immunoglobulin single variable domain(s) that is/are known to bind to said cell-associated antigen;
d) and optionally further evaluation, screening and/or selection of identified selection of fusion proteins from step b and/or c by epitope binning, epitope analysis, ligand competition assay, and/or functional assays;
e) and optionally producing said generated and/or identified second immunoglobulin single variable domain (or a polypeptide comprising said second immunoglobulin single variable domain),
f) and repeating steps above until an immunoglobulin single variable domain (or a polypeptide comprising said second immunoglobulin single variable domain) is obtained that is an inverse agonist of said cell-associated antigen.
   7. Immunoglobulin Single Variable Domains Obtainable by the Method: Epitope Walking with Multimeric Libraries

In the context of transmembrane proteins, and in particular proteins with multiple transmembrane domains, conformational epitopes, and in particular membrane-dependent conformational epitopes are of particular interest as targets for immunoglobulin single variable domains. For example, the pore of an ion channel represents a target of primary therapeutic importance. However, by use of conventional approaches, it is nearly impossible to generate immunoglobulin single variable domains that recognize such a target. The present invention provides for the generation of immunoglobulin single variable domains to such kind of conformational epitope.

The general principles of the present invention as set forth above will now be exemplified by reference to specific experiments. However, the invention is not to be understood as being limited thereto.

The entire contents of all of the references (including literature references, issued patents, published patent applications, and co-pending patent applications) cited throughout this application are hereby expressly incorporated by reference, particularly for the purposes cited herein.

### Experimental Part

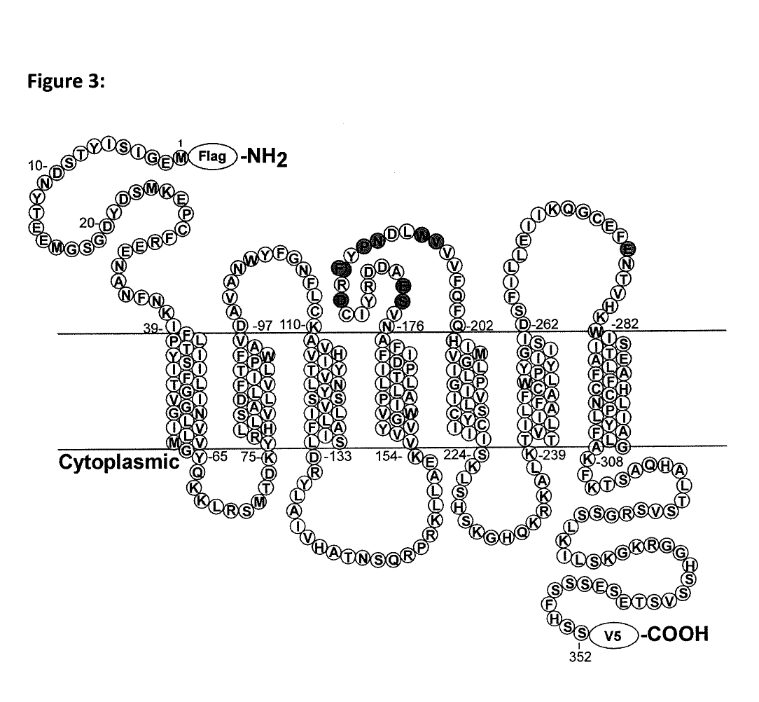

TABLE B-1
Sequences of human CXCR4 (CXCR4 Synonyms:
CXCR-4/Stromal cell-derived factor 1 receptor
(SDF-1 receptor)/Fusin/Leukocyte-derived seven
transmembrane domain receptor
(LESTR)/LCR1/FB22/NPYRL/HM89/CD184 antigen):

| | Clone name(s) used | SEQ ID |
|---|---|---|
| Amino acid sequence | in this document | NO: |
| MEGISSIPLPLLQIYTSDNYTEEMGSGDYDSMKEP | HUMAN | 1 |
| CFREENANFNKIFLPTIYSIIFLTGIVGNGLVILVMG | gi\|3059120\|emb\|CA | |
| YQKKLRSMTDKYRLHLSVADLLFVITLPFWAVD | A12166.1\| CXCR4 | |
| AVANWYFGNFLCKAVHVIYTVNLYSSVLILAFIS | [Homo sapiens]; | |
| LDRYLAIVHATNSQRPRKLLAEKVVYVGVWIPAL | human CXCR4- | |
| LLTIPDFIFANVSEADDRYICDRFYPNDLWVVVFQ | long; human | |
| FQHIMVGLILPGIVILSCYCIIISKLSHSKGHQKRKA | CXCR4_v3 | |
| LKTTVILILAFFACWLPYYIGISIDSFILLEIIKQGCE | | |
| FENTVHKWISITEALAFFHCCLNPILYAFLGAKFK | | |
| TSAQHALTSVSRGSSLKILSKGKRGGHSSVSTESE | | |
| SSSFHSS | | |
| MEGISIYTSDNYTEEMGSGDYDSMKEPCFREENA | Human CXCR4- | 6 |
| NFNKIFLPTIYSIIFLTGIVGNGLVILVMGYQKKLR | short, NM_003467.2 | |
| SMTDKYRLHLSVADLLFVITLPFWAVDAVANWY | (see also figure 3) | |
| FGNFLCKAVHVIYTVNLYSSVLILAFISLDRYLAIV | | |
| HATNSQRPRKLLAEKVVYVGVWIPALLLTIPDFIF | | |
| ANVSEADDRYICDRFYPNDLWVVVFQFQHIMVG | | |
| LILPGIVILSCYCIIISKLSHSKGHQKRKALKTTVILI | | |
| LAFFACWLPYYIGISIDSFILLEIIKQGCEFENTVHK | | |
| WISITEALAFFHCCLNPILYAFLGAKFKTSAQHAL | | |
| TSVSRGSSLKILSKGKRGGHSSVSTESESSSFHSS | | |
| MEGISIYTSDNYTEEMGSGDYDSMKEPCFREENA | CXCR4 mutant 1 | 19 |
| NFNKIFLPTIYSIIFLTGIVGNGLVILVMGYQKKLR | (F189V of human | |
| SMTDKYRLHLSVADLLFVITLPFWAVDAVANWY | CXCR4-short) | |
| FGNFLCKAVHVIYTVNLYSSVLILAFISLDRYLAIV | | |
| HATNSQRPRKLLAEKVVYVGVWIPALLLTIPDFIF | | |
| ANVSEADDRYICDRVYPNDLWVVVFQFQHIMVG | | |
| LILPGIVILSCYCIIISKLSHSKGHQKRKALKTTVILI | | |
| LAFFACWLPYYIGISIDSFILLEIIKQGCEFENTVHK | | |
| WISITEALAFFHCCLNPILYAFLGAKFKTSAQHAL | | |
| TSVSRGSSLKILSKGKRGGHSSVSTESESSSFHSS | | |
| MEGISIYTSDNYTEEMGSGDYDSMKEPCFREENA | CXCR4 mutant 2 | 20 |
| NFNKIFLPTIYSIIFLTGIVGNGLVILVMGYQKKLR | (V196E of human | |
| SMTDKYRLHLSVADLLFVITLPFWAVDAVANWY | CXCR4-short) | |
| FGNFLCKAVHVIYTVNLYSSVLILAFISLDRYLAIV | | |
| HATNSQRPRKLLAEKVVYVGVWIPALLLTIPDFIF | | |
| ANVSEADDRYICDRFYPNDLWEVVFQFQHIMVG | | |
| LILPGIVILSCYCIIISKLSHSKGHQKRKALKTTVILI | | |
| LAFFACWLPYYIGISIDSFILLEIIKQGCEFENTVHK | | |
| WISITEALAFFHCCLNPILYAFLGAKFKTSAQHAL | | |
| TSVSRGSSLKILSKGKRGGHSSVSTESESSSFHSS | | |
| MEGISIYTSDNYTEEMGSGDYDSMKEPCFREENA | CXCR4 mutant 3 | 21 |
| NFNKIFLPTIYSIIFLTGIVGNGLVILVMGYQKKLR | (D187V of human | |
| SMTDKYRLHLSVADLLFVITLPFWAVDAVANWY | CXCR4-short) | |
| FGNFLCKAVHVIYTVNLYSSVLILAFISLDRYLAIV | | |
| HATNSQRPRKLLAEKVVYVGVWIPALLLTIPDFIF | | |
| ANVSEADDRYICVRFYPNDLWVVVFQFQHIMVG | | |
| LILPGIVILSCYCIIISKLSHSKGHQKRKALKTTVILI | | |
| LAFFACWLPYYIGISIDSFILLEIIKQGCEFENTVHK | | |
| WISITEALAFFHCCLNPILYAFLGAKFKTSAQHAL | | |
| TSVSRGSSLKILSKGKRGGHSSVSTESESSSFHSS | | |
| MSIPLPLLQIYTSDNYTEEMGSGDYDSMKEPCFRE | NP_001008540, | 22 |
| ENANFNKIFLPTIYSIIFLTGIVGNGLVILVMGYQK | human CXCR4 | |
| KLRSMTDKYRLHLSVADLLFVITLPFWAVDAVA | isoform 2 | |
| NWYFGNFLCKAVHVIYTVNLYSSVLILAFISLDRY | | |
| LAIVHATNSQRPRKLLAEKVVYVGVWIPALLLTIP | | |
| DFIFANVSEADDRYICDRFYPNDLWVVVFQFQHI | | |
| MVGLILPGIVILSCYCIIISKLSHSKGHQKRKALKT | | |
| TVILILAFFACWLPYYIGISIDSFILLEIIKQGCEFEN | | |
| TVHKWISITEALAFFHCCLNPILYAFLGAKFKTSA | | |
| QHALTSVSRGSSLKILSKGKRGGHSSVSTESESSSF | | |
| HSS | | |

TABLE B-2
Sequences of reference compounds

| | | SEQ |
|---|---|---|
| | | ID |
| Amino Acid Sequence | Clone name | NO: |
| EVQLVESGGGLVQTGGSLRLSCAASGFTFSSYAM | 238D2 | 2 |
| SWVRQAPGKGLEWVSGIKSSGDSTRYAGSVKGR | | |
| FTISRDNAKNMLYLQMYSLKPEDTAVYYCAKSR | | |
| VSRTGLYTYDNRGQGTQVTVSS | | |
| EVQLMESGGGLVQAGGSLRLSCAASGRTFNNYA | 238D4 | 3 |
| MGWFRRAPGKEREFVAAITRSGVRSGVSAIYGDS | | |
| VKDRFTISRDNAKNTLYLQMNSLKPEDTAVYTC | | |
| AASAIGSGALRRFEYDYSGQGTQVTVSS | | |
| EVQLVESGGGLVQTGGSLRLSCAASGFTFSSYAM | 238D2-15GS- | 4 |
| SWVRQAPGKGLEWVSGIKSSGDSTRYAGSVKGR | 238D4 | |
| FTISRDNAKNMLYLQMYSLKPEDTAVYYCAKSR | | |
| VSRTGLYTYDNRGQGTQVTVSSGGGGSGGGGSG | | |
| GGGSEVQLMESGGGLVQAGGSLRLSCAASGRTF | | |
| NNYAMGWFRRAPGKEREFVAAITRSGVRSGVSAI | | |
| YGDSVKDRFTISRDNAKNTLYLQMNSLKPEDTA | | |
| VYTCAASAIGSGALRRFEYDYSGQGTQVTVSS | | |
| EVQLVESGGGLVQTGGSLRLSCAASGFTFSSYAM | 238D2-20GS- | 5 |
| SWVRQAPGKGLEWVSGIKSSGDSTRYAGSVKGR | 238D4 | |
| FTISRDNAKNMLYLQMYSLKPEDTAVYYCAKSR | | |
| VSRTGLYTYDNRGQGTQVTVSSGGGGSGGGGSG | | |
| GGGSGGGGSEVQLMESGGGLVQAGGSLRLSCAA | | |
| SGRTFNNYAMGWFRRAPGKEREFVAAITRSGVR | | |
| SGVSAIYGDSVKDRFTISRDNAKNTLYLQMNSLK | | |
| PEDTAVYTCAASAIGSGALRRFEYDYSGQGTQVT | | |
| VSS | | |
| EVQLVESGGGLVQPGNSLRLSCAASGFTFSSFGM | Alb-11 | 17 |
| SWVRQAPGKGLEWVSSISGSGSDTLYADSVKGRF | | |
| TISRDNAKTTLYLQMNSLRPEDTAVYYCTIGGSLS | | |
| RSSQGTLVTVSS | | |
| AAAEQKLISEEDLNGAAHHHHHH | Tag-1 or | 18 |
| | 3xFlag-His₆ | |

TABLE B-3
Linker sequences of the invention

| Name | SEQ | |
|---|---|---|
| of | ID | |
| linker | NO: | Amino acid sequences |
| 5GS | 7 | GGGGS |
| 6GS | 8 | SGGSGGS |
| 9GS | 9 | GGGGSGGGS |
| 10GS | 10 | GGGGSGGGGS |
| 15GS | 11 | GGGGSGGGGSGGGGS |
| 18GS | 12 | GGGGSGGGGSGGGGGGGS |
| 20GS | 13 | GGGGSGGGGSGGGGSGGGGS |
| 25GS | 14 | GGGGSGGGGSGGGGSGGGGSGGGGS |
| 30GS | 15 | GGGGSGGGGSGGGGSGGGGSGGGGSGGGGS |
| 35GS | 16 | GGGGSGGGGSGGGGSGGGGSGGGGSGGGGSG |
| | | GGGS |

TABLE B-4
Sequences of compounds of the invention

| | | |
|---|---|---|
| EVQLVESGGGLVQPGGSLRLSCAASGFTF | 238D2 with | 23 |
| SSYAMSWVRQAPGKGLEWVSGIKSSGDST | mutations: T14P, | |
| RYAGSVKGRFTISRDNAKNTLYLQMNSLR | M77T, Y82aN, | |
| PEDTAVYYCAKSRVSRTGLYTYDNRGQGT | K83R, and Q108L | |
| LVTVSS | | |
| EVQLVESGGGLVQPGGSLRLSCAASGRTF | 238D4 with | 24 |
| NNYAMGWFRQAPGKEREFVAAITRSGVRS | mutations: M5V, | |
| GVSAIYGDSVKDRFTISRDNAKNTLYLQM | A14P, R39Q, | |
| NSLRPEDTAVYYCAASAIGSGALRRFEYD | K83R, T91Y, | |
| YSGQGTLVTVSS | and Q108L | |
| EVQLVESGGGLVQAGGSLRLSCAASGRTF | 238D4 with | 25 |
| NNYAMGWFRRAPGKEREFVAAITRSGVRS | mutation M5V | |
| GVSAIYGDSVKDRFTISRDNAKNTLYLQM | | |
| NSLKPEDTAVYTCAASAIGSGALRRFEYD | | |
| YSGQGTQVTVSS | | |
| EVQLVESGGGLVQTGGSLRLSCAASGFTF | 238D2 with | 26 |
| SSYAMSWVRQAPGKGLEWVSGIKSSGDST | mutation M77T | |
| RYAGSVKGRFTISRDNAKNTLYLQMYSLK | | |
| PEDTAVYYCAKSRVSRTGLYTYDNRGQGT | | |
| QVTVSS | | |
| EVQLVESGGGLVQTGGSLRLSCAASGFTF | 238D2-20GS- | 27 |
| SSYAMSWVRQAPGKGLEWVSGIKSSGDST | 238D4(M5V) | |
| RYAGSVKGRFTISRDNAKNMLYLQMYSLK | | |
| PEDTAVYYCAKSRVSRTGLYTYDNRGQGT | | |
| QVTVSSGGGGSGGGGSGGGGSGGGGSEVQ | | |
| LVESGGGLVQAGGSLRLSCAASGRTFNNY | | |
| AMGWFRRAPGKEREFVAAITRSGVRSGVS | | |
| AIYGDSVKDRFTISRDNAKNTLYLQMNSL | | |
| KPEDTAVYTCAASAIGSGALRRFEYDYSG | | |
| QGTQVTVSS | | |
| EVQLVESGGGLVQTGGSLRLSCAASGFTF | 238D2(M77T)- | 28 |
| SSYAMSWVRQAPGKGLEWVSGIKSSGDST | 20GS-238D4(M5V) | |
| RYAGSVKGRFTISRDNAKNTLYLQMYSLK | | |
| PEDTAVYYCAKSRVSRTGLYTYDNRGQGT | | |
| QVTVSSGGGGSGGGGSGGGGSGGGGSEVQ | | |
| LVESGGGLVQAGGSLRLSCAASGRTFNNY | | |
| AMGWFRRAPGKEREFVAAITRSGVRSGVS | | |
| AIYGDSVKDRFTISRDNAKNTLYLQMNSL | | |
| KPEDTAVYTCAASAIGSGALRRFEYDYSG | | |
| QGTQVTVSS | | |
| EVQLVESGGGLVQPGGSLRLSCAASGFTF | 238D2(T14P, | 29 |
| SSYAMSWVRQAPGKGLEWVSGIKSSGDST | M77T,Y82aN, | |
| RYAGSVKGRFTISRDNAKNTLYLQMNSLR | K83R,Q108L)- | |
| PEDTAVYYCAKSRVSRTGLYTYDNRGQGT | 20GS-238D4 | |
| LVTVSSGGGGSGGGGSGGGGSGGGGSEVQ | (M5V) | |
| LVESGGGLVQAGGSLRLSCAASGRTFNNY | | |
| AMGWFRRAPGKEREFVAAITRSGVRSGVS | | |
| AIYGDSVKDRFTISRDNAKNTLYLQMNSL | | |
| KPEDTAVYTCAASAIGSGALRRFEYDYSG | | |
| QGTQVTVSS | | |
| EVQLVESGGGLVQPGGSLRLSCAASGFTF | 238D2(T14P, | 30 |
| SSYAMSWVRQAPGKGLEWVSGIKSSGDST | M77T,Y82aN, | |
| RYAGSVKGRFTISRDNAKNTLYLQMNSLR | K83R,Q108L)- | |
| PEDTAVYYCAKSRVSRTGLYTYDNRGQGT | 20GS-238D4 | |
| LVTVSSGGGGSGGGGSGGGGSGGGGSEVQ | (M5V, A14P, | |
| LVESGGGLVQPGGSLRLSCAASGRTFNNY | R39Q, K83R, | |
| AMGWFRQAPGKEREFVAAITRSGVRSGVS | T91Y,Q108L) | |
| AIYGDSVKDRFTISRDNAKNTLYLQMNSL | | |
| RPEDTAVYYCAASAIGSGALRRFEYDYSG | | |
| QGTLVTVSS | | |

### Example 1

### Isolation of Monovalent Anti-CXCR4 Nanobodies

### Example 1.1

### Antigen Preparation

cDNA—pcDNA3.1-CXCR4 was obtained as gift from Dr. Tensen (Leiden University Medical Center, Leiden, The Netherlands).
Cell culture and transfection—HEK293T, CHO and COS-7 cells were maintained at 37° C. in a humidified 5% CO₂, 95% air atmosphere in Dulbecco's modified Eagle's medium (DMEM) containing 2 mM L-glutamine, 50 IU/ml penicillin, 50 μg/ml streptomycin, and 10% (v/v) fetal calf serum. Cells were transiently transfected with a constant amount of total DNA using DEAE-dextran or linear 25 kDa polyethyleneimine (Polysciences, Warrington, Pa.) as carrier as previously described (Verzijl et al., Noncompetitive Antagonism and Inverse Agonism as Mechanism of Action of Nonpeptidergic Antagonists at Primate and Rodent CXCR3 Chemokine Receptors. Journal of Pharmacology and Experimental Therapeutics (2008) 325 (2):544-55).
Preparation of membrane fractions—Membranes from HEK293T, CHO and COS-7 cells transiently expressing CXCR4 were prepared 48 h after transfection as follows. Cells were washed and scraped from the cell culture dishes with ice-cold PBS containing 1 mM EDTA. The scraped cells were pelleted at 1500×g for 10 mM at 4° C. The pellet was washed and then resuspended in ice-cold membrane buffer (15 mM Tris, pH 7.5, 1 mM EGTA, 0.3 mM EDTA, and 2 mM MgCl₂). The cell suspension was homogenized by 10 strokes at 1200 rpm using a Teflon-glass homogenizer and rotor and further subjected to three freeze-thaw cycles using liquid nitrogen. Membranes were separated by centrifugation at 40,000 g for 25 min at 4° C. The membrane pellet was washed and resuspended in ice-cold Tris-sucrose buffer (20 mM Tris, pH 7.4, and 250 mM sucrose) and frozen in liquid nitrogen. The total protein was determined using a Bradford assay (Bio-Rad).

### Example 1.2

### Immunizations

For immunization, HEK293 (human embryonic kidney) cells transiently expressing human CXCR4 were used as “antigen”.

Two llamas were immunized according to standard protocols with 6 single injections of cells (1-4*10E7 cells) at day 0, 7, 21, 32, 43 and 56. Blood samples were collected from these animals 4 and 8 days after the 6^{th} injections.

### Example 1.3

### Library Construction

Peripheral blood mononuclear cells were prepared from blood samples using Ficoll-Hypaque according to the manufacturer's instructions. Next, total RNA was extracted from these cells and used as starting material for RT-PCR to amplify the Nanobody encoding genes. The resulting PCR-fragments were cloned into phagemid vector pAX50. Phages were prepared according to standard methods (see prior art and applications filed by applicant cited herein) and stored at 4° C. for further use. Two phage libraries, 217 and 218, one from each llama, were generated.

### Example 1.4

### Phage Display Selection

To identify Nanobodies recognizing CXCR4, phage libraries 217 and 218 were used in a phage display selection on membrane preparations of cells overexpressing hCXCR4. Two rounds of selection were performed using a membrane preparation from CXCR4-expressing CHO cells in round one and a membrane preparation from CXCR4-expressing COST cells in round two.

For the selection the antigen-containing cell membrane preparation was coated onto Maxisorp plates (Nunc) overnight at 4° C. (100 μg/ml in PBS) and blocked with 4% Marvel-PBS for 1 hour. Phages in 1% Marvel-PBS were incubated with the coated antigen-membrane in the presence of a membrane preparation from CHO cells transfected with a non-relevant GPCR. After 2 hours incubation, the plates were washed extensively with PBS. Bound phages were eluted using trypsin (1 mg/ml) for 15 min at RT and rescued and reamplified in E. coli TG1.

Outputs from round two were analyzed for enrichment factors (# phages in eluate relative to controls) and outputs with highest enrichment factors were chosen for further analysis. For this the polyclonal phage pool was rescued in E. coli TG1 and E. coli cells were plated onto agar plates. Individual TG1 colonies were picked and grown in 96 deep well plates (1 ml volume) to produce monoclonal phages (by adding helper phage) or periplasmic extracts containing soluble Nanobodies (by adding IPTG). Periplasmic extracts (volume ˜90 μl) were prepared according to standard methods (see prior art and applications filed by applicant cited herein).

### Example 1.6

### Phage ELISA Binding Assay

Binding specificity of the Nanobodies was assessed in a phage ELISA binding assay. In short, membrane preparations of CHO cells transfected either with CXCR4 or with a non-relevant GPCR (control) were coated overnight at 4° C. directly onto Maxisorp microtiter plates at 20 μg/ml in PBS. Free binding sites were blocked using 4% Marvel-PBS for 1 h. 15 μl of monoclonal phage preparations were mixed with 100 μl 1% Marvel-PBS and incubated with the coated membrane preparations for 2 hours. After extensive washing with PBS, phage binding was detected using an anti-M13-HRP antibody conjugate. Specific binding to CXCR4 was determined based on binding signal over the control.

### Example 1.7

### Screening of CXCR4-Binding Nanobodies by Displacement of [¹²⁵I]-CXCL12

[¹²⁵I]-labelling-[¹²⁵I]-labelled CXCL12 (2,200 Ci/mmol) was obtained from Analytical Sciences (Boston, Mass.). Radiolabeling of Nanobodies with ¹²⁵I was performed using the Iodo-gen method (Pierce, Rockford, Ill.) according to the manufacturer's protocol. ¹²⁵I-labeled Nanobodies were separated from free iodine (>99%) using a Sephadex G-25 gel filtration column (Amersham Biosciences, Piscataway, N.J.). Iodine incorporation and specific activity were controlled via precipitation of the protein with trichloroacetic acid.

Competition binding assay—Periplasmic fractions were screened in a competition binding assay using membranes from HEK293T cells transiently expressing CXCR4. In short, periplasmic extracts (1:10) or ligands were pre-incubated with membranes in binding buffer (50 mM HEPES (pH 7.4), 1 mM CaCl₂, 5 mM MgCl₂, 100 mM NaCl, 0.5% bovine serum albumin) supplemented with 0.5% BSA for 1 h at 22° C. before the addition of [¹²⁵I]-CXCL12 (40 pM) or [¹²⁵I]-238D2 (3 nM) or [¹²⁵I]-238D4 (3 nM) for additional 2 h at 22° C. Non-specific binding was determined in the presence of AMD3100 (3 μM, Sigma Aldrich). Membranes were then harvested over polyethylenimine (0.5%)-treated Whatman GF/C filter plates and washed three times with ice cold binding buffer containing 500 mM NaCl. Plates were counted by liquid scintillation.

No inhibition was observed for control periplasmic extracts containing Nanobodies directed against membrane proteins different from CXCR4. All primary hits were confirmed in a second screen and the nanobody encoding DNA of CXCL12-displacing Nanobody-producing clones were sequenced. Sequencing analysis identified Nanobodies 238D2 and 238D4 as strongly displacing [¹²⁵I]-CXCL12.

Characterization of Nanobody binding to CXCR4—Following purification, receptor binding to characteristics for 238D2 and 238D4 were investigated on cell membranes from HEK293T cells transiently expressing CXCR4. The Nanobodies 238D2 and 238D4 fully displace all specifically bound [¹²⁵I]-CXCL12 and show functional antagonist affinities (Kᵢ) to CXCR4 in the low nanomolar range. Both Nanobodies also compete for binding to CXCR4 as shown by the full displacement of [¹²⁵I]-238D2 by 238D4 and of [¹²⁵I]-238D4 by 238D2. Furthermore, the small molecule ligand AMD3100 displaces [¹²⁵I]-238D2 and [¹²⁵I]-238D4 with affinities comparable to those obtained against [¹²⁵I]-CXCL12 indicating that AMD3100 competes with the Nanobodies 238D2 and 238D4 for the same receptor. The monoclonal antibody 12G5 that has previously been reported to label a certain subpopulation of CXCR4 (J. Virol. Baribaud et al. 75 (19): 8957) potently but incompletely displaces specifically bound [¹²⁵I]-CXCL12, [¹²⁵I]-238D2 and [¹²⁵I]-238D4 from CXCR4 (Table C-1).

TABLE C-1
Receptor affinity (pKᵢ) and maximal displacement of [¹²⁵I]-CXCL12, [¹²⁵I]-238D2 and [¹²⁵I]-
238D4 for monovalent Nanobodies and CXCR4 reference ligands. The experiments were
performed on membranes from HEK293T cells transiently expressing CXCR4. Data were
shown as means ± S.E.M. The number of experiments is given as n.

| | [¹²⁵I]-CXCL12 | | | [¹²⁵I]-238D2 | | | [¹²⁵I]-238D4 | | |
|---|---|---|---|---|---|---|---|---|---|
| | Displace | | | Displace | | | Displace | | |
| | m. (%) | pKᵢ | n | m. (%) | pKᵢ | n | m. (%) | pKᵢ | n |
| 238D2 | 93 ± 5 | 8.01 ± 0.12 | 6 | 97 ± 6 | 8.41 ± 0.11 | 4 | 105 ± 4 | 8.23 ± 0.23 | 4 |
| 238D4 | 99 ± 5 | 8.22 ± 0.16 | 6 | 101 ± 1 | 8.80 ± 0.23 | 4 | 103 ± 1 | 8.55 ± 0.09 | 4 |
| CXCL12 | 105 ± 2 | 9.84 ± 0.13 | 3 | 98 ± 8 | 7.46 ± 0.17 | 4 | 93 ± 2 | 7.45 ± 0.12 | 4 |
| AMD3100 | 94 ± 2 | 7.41 ± 0.28 | 3 | 102 ± 1 | 7.74 ± 0.19 | 4 | 99 ± 4 | 7.34 ± 0.16 | 4 |
| 12G5 | 54 ± 5^{a} | 9.19 ± 0.19 | 3 | 89 ± 2^{a} | 9.65 ± 0.17 | 4 | 90 ± 1^{a} | 9.31 ± 0.16 | 4 |

^{a}Significantly different from 100%.

Data analysis and presentation—Data are presented as mean±S.E.M. from n independent experiments. Concentration response curves (E/[A] curves) were fitted to the Hill equation using an iterative, least-squares method (GraphPad Prism 4.0, GraphPad Software, San Diego, Calif.) to provide maximal inhibitory effects (Iₘₐₓ), half maximal effective (EC₅₀) or inhibitory concentrations (IC₅₀). Competition binding affinities and functional antagonist affinities (Kᵢ) were calculated using the Cheng and Prusoff equation Kᵢ=IC₅₀/(1+[agonist]/EC₅₀) (Cheng & Prusoff, 1973).

Results were compared using Student's t-test or one way analysis of variance followed by Bonferroni corrected t-test for stepwise comparison, when multiple comparisons were made. P values <0.05 were considered to be significant.

CXCR4—specific Nanobodies behave as neutral antagonists or inverse agonists on constitutively active mutants of CXCR4—The CXCR4—specific monovalent Nanobodies 238D2 and 238D4 as well as their bivalent fusion products L3 and L8 were investigated on the constitutively active CXCR4 mutant N119A (equivalent to N3.35A in the Ballestros-Weinstein numbering of class A GPCRs). Mutants of N119 have previously been identified by Peiper and co-workers as the only mutants which have been selected from a CXCR4 random mutagenesis library using a yeast reporter gene assay for constitutively active mutants (CAMs) (Zhang W. B., Navenot J. M., Haribabu B., Tamamura H., Hiramatu K., Omagari A., Pei G., Manfredi J. P., Fujii N., Broach J. R., Peiper S. C. (2002). A point mutation that confers constitutive activity to CXCR4 reveals that T140 is an inverse agonist and that AMD3100 and ALX40-4C are weak partial agonists. J. Biol. Chem. 277:24515-24521). Despite of the large number of CAMs for other class A GPCRs and further efforts to generate additional CAMs for CXCR4 (Berchiche et al., 2007), the N119 mutants of CXCR4 remain the only known CAMs for this receptor. Both monovalent Nanobodies investigated, 238D2 and 238D4 as well as their bivalent fusion products L3 and L8, were able to bind to CXCR4 (N119A). Their binding affinities were somewhat less compared to the wild type receptor. Due to the reduced affinity, no plateau was reached at the highest nanobody test concentration of 2 μM.
Methods: Preparation of membranes and competition binding experiments with [125I]-CXCL12 (40 pM) were performed as described before for wild type CXCR4 (vide supra).

The functional profile of monovalent Nanobodies 238D2 and 238D4 as well as their bivalent fusion products L3 and L8 on CXCR4 (N119A) were investigated by measurement of the ligand-induced alteration of the basal inositol phosphate accumulation. HEK293T cells transiently expressing CXCR4 (N119A) show a 3-8 times higher basal rate of inositol phosphate accumulation compared to wild type CXCR4 or mock (which are virtually at the same level). The ability of CXCL12 to further stimulate the mutant receptor is reduced (0.4 fold over basal) compared to wild type. 238D4 and bivalent variants of 238D4 (i.e. L3=238D2-15GS-238D4, SEQ ID NO: 4; and L8=238D2-20GS-238D4, SEQ ID NO: 5) behave as partial inverse agonists at this mutant and reduce the constitutively increased basal signalling of CXCR4 (N119A) by 49, 64, and 65%, respectively. The nanobody-induced reduction of basal inositol phosphate accumulation was antagonized by the selective neutral CXCR4 antagonist plerixafor confirming that the observed inverse antagonistic effects are mediated via CXCR4 (N119A). Only antagonistic but no significant inverse agonistic activities were observed for 238D2 and plerixafor although these ligands clearly bind to the mutant receptor.

Our results show that Nanobodies can act as neutral antagonists or inverse agonists on constitutively active CXCR4 mutants. A significant number of the top selling GPCR drugs behave as inverse agonists rather than neutral antagonists (Milligan G. (2003). Constitutive activity and inverse agonists of G protein-coupled receptors: a current perspective. Mol. Pharmacol. 64:1271-1276) and it has been claimed that inverse agonists may have specific therapeutic benefits compared with neutral antagonists for several diseases including cancer (Kenakin T (2004). Efficacy as a vector: the relative prevalence and paucity of inverse agonism. Mol. Pharmacol. 65:2-11). Despite of the novelty of our observation that CXCR4-specific nanobodies may behave as inverse agonists, the physiological relevance of inverse to CXCR4 agonism is not clear. As we could not detect any significant basal activity of CXCR4 (wt) compared to mock in the inositol phosphate accumulation assay, it is impossible to detect any inverse agonism at least in this assay. Furthermore, the most obvious function of CXCR4 is the chemotactic recruitment of stem cells to the bone marrow. The chemotaxis is mediated by an asymmetric activation of cell surface receptors to let cells migrate towards a chemoattractant gradient. Thus, chemotaxis is strictly dependent on a chemoattractant ligand. However, inverse agonists may be superior over neutral antagonists to inhibit other functions of CXCR4 like chemokinesis or promotion of tumour growth. Furthermore, an inverse agonists to CXCR4 may be superior if not required over neutral antagonists in the treatment of the WHIM syndrome that is an immunodeficiency disease characterized by neutropenia, hypogammaglobulinemia and extensive human papillomavirus (HPV) infection. Hernandez et al (Nature Genetics 34, 70-74 (2003)) described the localization of the gene associated with WHIM syndrome to a region of roughly 12 cM on chromosome 2q21 and the identification of truncating mutations in the cytoplasmic tail domain of the gene encoding chemokine receptor 4 (CXCR4) indicating that CXCR4 may have basal activity and thus an agent with an inverse agonistic effect may be superior.

### Example 1.8

### Shotgun Mutagenesis Epitope Mapping of Nanobodies 238D2 and 238D4

Method: See e.g. Willis, et al. 2008. Virus-like particles as quantitative probes of membrane protein interactions. Biochemistry 47:6988-6990. In short, the method comprises the following steps:

Create comprehensive mutation library
Start with a cDNA in eukaryotic expression plasmid
Create library where every amino acid is individually mutated

Express each clone within living cells in 384-well microplates

Detect structure and/or function of proteins

### Materials:

Parental plasmid: Human CXCR4-short (SEQ ID NO: 6, 352 amino acids)
Epitope tags: N-terminal Flag (to detect surface expression), C-terminal V5 (to detect full-length translation)
Cell types used for immunofluorescence assays: HEK-293T

### Results:

A library with the CXCR4 mutants was generated. Table C-2 describes the library statistics that were obtained.

TABLE C-2
CXCR4 Mutation Library Statistics

| | |
|---|---|
| Total number of clones in library | 731 |
| CXCR4 AA residues mutated | 352 of 352 |
| Average number of AA mutations per clone | 1.3 |
| Average number of mutations per AA residue | 2.7 |
| Number (percentage) of AA mutated at least once | 352 (100%) |
| Number (percentage) of AA mutated at least twice | 348 (98.9%) |
| Number (percentage) of clones containing a single AA | 557 (76.2%) |
| mutation | |
| Number (percentage) of clones containing two AA | 146 (19.9%) |
| mutations | |
| Number (percentage) of clones containing more than two | 28 (3.8%) |
| AA mutations | |

The immunofluorescence assay conditions (to detect binding) were optimized for the nanbodies to be tested. In short, 384-well immunofluorescence (IF) assay using HEK-293T cells transiently was transfected with different concentrations of CXCR4 DNA. The plate was first incubated with different concentrations of either Nanobody 238D2 or 238D4, then with 9E10, and finally with Cy3. The commercially-available anti-CXCR4 MAb 12G5 was used as an IF control. It was found that the CXCR4 mutation library is best screened using 1 ug/ml of Nanobody 238D2 and 1 ug/ml Nanobody 238D4.

Mapping Nanobody Binding to CXCR4 Mutation Library: The entire library was screened in triplicate with each Nanobody. The data was background-subtracted and normalized to wild type reactivity and averaged across the repeats. Nanobody binding was plotted as a function of average surface expression, and measured by immunoreactivity of an N-terminal Flag epitope tag. Thresholds of 60% Flag and 30% Nanobody were used to identify critical clones. Using standard IF conditions, we identified 1 critical residue (2 critical clones). Using increased stringency assay conditions (high salt), we identified 5 additional residues (Tables C-3 and C-4).

TABLE C-3
Data (in % immunofluoresence) Table of Critical Residues Identified for
Nanobody 238D2 and 238D4. Results from 3 independent immunofluorescence experiments
are shown (+/−range in parentheses). Critical amino acids in clones containing more than one
mutation were differentiated by comparing the reactivity of other clones with mutation of the
same residues. Critical amino acids for Nanobody 238D2 were identified under high
stringency assay conditions (mutations of F189 affect binding of 238D2 at both high and low
stringency).

| | | | | | Nanobody | Nanobody | |
|---|---|---|---|---|---|---|---|
| Residue | Clone | Mutation | V5 | Flag | 23802 | 23804 | 12G5 |
| S178 | 2396 | S178C | 79.1 (22.7) | 66.5 (21.6) | 44.9 (13.4) | 28.2 (14.0) | 91.8 (16.7) |
| | 709 | P163L, S178I | 130.2 (30.9) | 81.2 (26.9) | 64.1 (19.6) | 30.0 (24.0) | 113.0 (41.4) |
| | 1057 | K230E, S178R | 83.9 (8.3) | 95.2 (14.0) | 54.6 (5.8) | 11.4 (20.1) | 111.6 (41.3) |
| E179 | 414 | C218R, E179V, S351T | 32.2 (17.3) | 95.8 (17.4) | 71.1 (6.7) | 8.0 (20.4) | −9.1 (24.5) |
| | 2834 | E179V, G258E | 62.4 (16.3) | 87.1 (8.3) | 84.6 (7.8) | 19.5 (21.6) | 1.5 (5.8) |
| D187 | 3728 | D187A, Y157C | 95.0 (7.3) | 92.6 (16.5) | 77.1 (18.2) | −16.4 (14.8) | 95.1 (30.0) |
| | 2084 | D187V | 121.9 (20.4) | 94.71 (17.2) | 93.6 (5.2) | −8.0 (7.6) | 89.5 (10.6) |
| F189 | 1129 | F189V | 72.4 (33.1) | 96.1 (22.8) | −1.9 (24.2) | 5.2 (27.1) | 94.7 (16.7) |
| | 913 | F189S, K308R | 108.1 (10.7) | 96.8 (35.1) | −32.6 (18.7) | −16.9 (19.9) | 110.5 (28.5) |
| | 465 | F189L, S319P, V155E | 105.7 (9.2) | 53.0 (39.62) | 31.8 (16.2) | 12.2 (18.9) | 50.4 (25.6) |
| P191 | 2184 | P191T | 127.6 (22.0) | 107.4 (16.0) | 15.8 (5.4) | 123.9 (21.1) | 117.9 (26.8) |
| N192 | 914 | N192K | 60.9 (10.5) | 68.1 (8.4) | 8.3 (3.6) | 55.8 (12.7) | 57.7 (51.7) |
| W195 | 3630 | W195R | 102.1 (6.0) | 92.4 (16.9) | 10.0 (10.4) | 58.8 (7.7) | 85.5 (26.6) |
| V196 | 2270 | V196E | 131.7 (22.0) | 92.8 (16.5) | 20.6 (32.9) | 94.0 (23.8) | 96.8 (11.7) |
| E277 | 185 | E277A, I245V, N298S | 111.0 (24.8) | 91.6 (11.7) | 35.5 (12.3) | 69.3 (36.9) | 93.9 (22.1) |
| | 3622 | E277G | 151.7 (23.7) | 73.2 (11.3) | 19.5 (11.9) | 52.5 (20.4) | 61.3 (34.9) |

TABLE C-4
Relative ranking of amino acids (most to least critical).

| | 238D2 | | 238D4 | |
|---|---|---|---|---|
| Relative ranking | F189 | −0.9 | D187 | −12.2 |
| of amino acids | N192 | 8.3* | F189 | 0.2 |
| (most to least | W195 | 10.0* | E179 | 13.8 |
| critical) | P191 | 15.8* | S178 | 23.2 |
| | V196 | 20.6* | | |
| | E277 | 27.5* | | |

The critical amino acids identified for each Nanobody are listed in relative order of importance to the interaction, from most (top) to least (bottom) critical, based on relative nanobody reactivity with clones containing a mutation at the critical residue. The average nanobody reactivity for each amino acid is listed next to each residue (averaging the values for all clones containing a mutation at that residue). Values marked with an asterisk (*) were identified under high stringency conditions.

### Example 1.9

### Footprint Assay to Determine Epitope A and/or Epitope B Binders According to the Invention

Further to the displacement assay and analysis as disclosed in example 1.7 supra, it was found that the many of the anti-CXCR4 Nanobodies competed with CXCL12 and AMD3100, similar to 238D2 and 238D4. Further we wanted to delineate this group into “238D2-like” or “epitope A binders” and “238D4-like” or “epitope B binders” by a limited epitope mapping effort.

Based on the data from example 1.8, we introduced 3 single point-mutations in ECL2 of CXCR4 that allowed to discriminate between 238D2 and 238D4-like epitopes: V196E (specifically disturbing D2 binding)-SEQ ID NO: 20, D187V (specifically disturbing D4 binding)-SEQ ID NO: 21, F189V (disturbing binding of both D2 and D4)-SEQ ID NO: 19. The mab 12G5 was binding to all point-mutants and thus served as a control for membrane expression (see Table B-3). For the epitope mapping, transient transfections of the three CXCR4 mutants and wildtype human CXCR4 in the pcDNA3.1 vector were done in Hek293T cells, after which Nanobody binding of different families was assessed by flow cytometry using detection of the Myc-tag, followed by secondary anti-mouse PE. Two concentrations of Nanobody were tested, 10 nM and 100 nM, and experiment was repeated with essentially the same results. Binding of the Nanobodies to HEK293T hCXCR4 cells was used for normalization using the following formulas:

$$Ratio 12 G 5 mAb = \frac{Binding mutant CXCR 4}{Binding hCXCR 4}$$

$$\% Binding = ( 1 - ( \frac{\begin{matrix}( Binding hCXCR 4 * ratio 12 G 5 mAb ) - \\ Binding mutant CXCR 4 )\end{matrix}}{( Binding hCXCR 4 * ratio 12 G 5 mAb )} ) ) * 100$$

Percentage of binding to the mutant receptors was calculated for each Nanobody concentration, and a position was considered as critical when less than 25% residual binding (average of n=3) was observed (Table C-5). For some Nanobodies partial loss of binding was observed (between 25 and 75%), which may indicate that the introduced mutation was tolerated but still positioned within the footprint. Nanobodies could be grouped according to their different binding patterns, not only in “238D2-like” or “epitope A binders” and “238D4-like” or “epitope B binders” but also into other new groups.

TABLE C-5
Footprint analysis of 44 different CXCR4-specific Nanobodies for binding to
mutant CXCR4 receptors expressed on Hek293T cells. Average percentage binding was
determined (n = 3).

| Clone | Epitope | HEK293T | HEK293T | HEK293T | Hek293T | Footprint |
|---|---|---|---|---|---|---|
| ID | group | CXCR4 | D187V | F189V | V196E | (loss of binding) |
| NB1 | A | + | + | − | − | F189-V196 |
| NB2 | A | + | + | − | − | F189-V196 |
| NB3 | A | + | + | − | − | F189-V196 |
| 238D2 | A | + | + | − | − | F189-V196 |
| NB4 | A | + | + | − | − | F189-V196 |
| NB5 | A | + | + | − | − | F189-V196 |
| NB6 | B | + | − | − | + | D187-F189 |
| 238D4* | B | + | − | − | + | D187-F189 |
| NB7 | B | + | − | − | + | D187-F189 |
| NB8 | C | + | − | + | − | D187-V196 |
| NB9 | C | + | − | + | − | D187-V196 |
| NB10 | D | + | − | +/− | − | D187-partF189-V196 |
| NB11 | D | + | − | +/− | − | D187-partF189-V196 |
| NB12 | E | + | − | − | − | D187-F189-V196 |
| NB13 | E | + | − | − | − | D187-F189-V196 |
| NB14 | E | + | − | − | − | D187-F189-V196 |
| NB15 | E | + | − | − | − | D187-F189-V196 |
| NB16 | E | + | − | − | − | D187-F189-V196 |
| NB17 | E | + | − | − | − | D187-F189-V196 |
| NB18 | E | + | − | − | − | D187-F189-V196 |
| NB19 | E | + | − | − | − | D187-F189-V196 |
| NB20 | E | + | − | − | − | D187-F189-V196 |
| NB21 | E | + | − | − | − | D187-F189-V196 |
| NB22 | E | + | − | − | − | D187-F189-V196 |
| NB23 | E | + | − | − | − | D187-F189-V196 |
| NB24 | E | + | − | − | − | D187-F189-V196 |
| NB25 | E | + | − | − | − | D187-F189-V196 |
| NB26 | F | + | +/− | − | − | partD187-F189-V196 |
| NB27 | F | + | +/− | − | − | partD187-F189-V196 |
| NB28 | G | + | + | + | − | V196 |
| NB29 | G | + | + | + | − | V196 |
| NB30 | H | + | +/− | + | − | partD187-V196 |
| NB31 | H | + | +/− | + | − | partD187-V196 |
| NB32 | I | + | − | + | + | D187 |
| NB33 | J | + | − | +/− | +/− | D187-partF189-partV196 |
| NB34 | K | + | + | − | + | F189 |
| NB35 | K | + | + | − | + | F189 |
| NB36 | K | + | + | − | + | F189 |
| NB37 | K | + | + | − | + | F189 |
| NB38 | L | + | + | − | +/− | F189-part V196 |
| NB39 | L | + | + | − | +/− | F189-part V196 |
| NB40 | M | + | +/− | − | +/− | partD187-F189-part V196 |
| NB41 | M | + | +/− | − | +/− | partD187-F189-part V196 |
| NB42 | N | + | + | + | + | — |

| | | | | |
|---|---|---|---|---|
| 12G5 mab | + | + | + | + |

Legend:
+ >75% binding to mutant rel. to hCXCR4
+/− >25 <=75% binding to mutant rel. to hCXCR4
− <=25% binding to mutant rel. to hCXCR4
238D4* A family member of 238D4 was used

### Example 2

### Generation and Screening of Nanobody-Fusion Libraries

### Example 2.1

### Vector Design

Vectors pAX141 and pAX142 are designed to facilitate phage display of a fusion protein consisting of two Nanobodies. Both vectors are derived from vector pAX50, which is a derivative of pUC119 and contains the following features: a LacZ promoter, a M13 phage gIII protein coding sequence, an ampicillin resistance gene, a multiple cloning site (MCS) and a hybrid gIII-pelB leader sequence. The gene of interest is cloned in frame and upstream of a c-myc tag and a (His)6 tag for purification and detection.

To generate vectors pAX141 and pAX142 the MCS of pAX50 is modified to allow for the insertion of two Nanobody genes in frame with the C-terminally fused phage gIII protein. Nanobody genes are inserted at the N-terminal position using restriction sites MfeI and BspEI and at the central position using restriction sites BamHI and BstEII. To facilitate cloning via BamHI a BamHI site in gIII is eliminated. Vector pAX141 encodes for a Gly₄SerGly₃Ser (9GS) spacer and vector pAX142 encodes for a (Gly₄Ser)₅ (25GS) spacer linking the two Nanobody building blocks.

For production of soluble Nanobodies after selection Nanobody genes are cloned into E. coli expression vector pAX100. pAX100 is derived from pUC119 and contains a LacZ promoter, a kanamycin resistance gene, a multiple cloning site, an OmpA leader sequence, a C-terminal c-myc tag and a (His)₆ tag in frame with the Nanobody sequence.

Individual Nanobody genes are first amplified via PCR to introduce MfeI and BstEII restriction sites at the 5′- and 3′-end, respectively, for subcloning into pAX100. Genes of fusion proteins of two Nanobodies are directly excised from pAX141 or pAX142 via MfeI and BstEII restriction sites and the resulting DNA fragments are inserted into pAX100 for production of soluble Nanobody fusion proteins.

### Example 2.2

### Verification of Display of Functional Nanobody-Fusion Proteins on pIII of Phage

The functional display of Nanobody-fusion proteins on phage particles is confirmed using the following constructs:
1) Dummy-9GS/25GS-238D4-gIIIp
2) 238D2-9GS/25GS-238D4-gIIIp
3) 238D4-gIIIp
4) 238D2-gIIIp

Dummy=functional Nanobody not recognizing CXCR4

238D2 and 238D4=anti CXCR4 Nanobodies

The genes encoding for the individual Nanobodies of constructs 1) to 4) are amplified via PCR introducing the necessary restriction sites for insertion into the N-terminal (MfeI and BspEI) or central (BamHI and BstEII) fusion protein position. PCR fragments are digested with the appropriate restriction enzymes and inserted into the identically linearised vectors pAX141 and pAX142.

Monoclonal phages displaying constructs 1) to 4) are produced and the binding characteristics are assessed in a phage ELISA binding assay. Wells are coated with either CXCR4+ or CXCR4− lipoprotein particles (Integral Molecular, Inc., Philadelphia, Pa.) or the Dummy antigen. After blocking with Marvel-PBS, phages in Marvel-PBS are added to the wells as dilution series. Addition of no phages or phages displaying an irrelevant Nanobody is used as negative control. Phage binding is detected using an anti-M13-HRP antibody conjugate.

### Example 2.3

### Construction of Nanobody-Fusion Libraries

For the generation of Nanobody-fusion libraries Nanobody 238D4 is inserted into pAX141 and pAX142 at the central position using restriction sites BamH1 and BstEII. For this two changes are introduced into the original sequence of clone 238D4. First, an internal BspEI restriction site is deleted in the central 238D4 building block facilitating depletion of library 218 of clone 238D4 during the cloning process. Second, Methionine at position 5 is mutated to the canonical Valine to make the sequence compatible with standard Nanobody primers.

For insertion at the N-terminal position Nanobody library 218 (and possibly library 217) are PCR amplified, digested using restriction sites MfeI and BspEI and cloned into pAX141-238D4 and pAX142-238D4 vectors, respectively. Phages are prepared according to standard methods (see prior art and applications filed by applicant cited herein) and stored at 4° C. for further use.

### Example 2.4

### Phage Display Selection of Nanobody-Fusion Libraries

Nanobody-fusion phage libraries are used in a phage display selection against CXCR4+ lipoprotein particles. For the selection microtiter plate wells are coated with CXCR4+ or CXCR4− particles (null) at different concentrations or not coated (NC). After blocking of the wells with 4% Marvel/PBS phages in 2% Marvel-PBS are added to the wells and incubated for 2 to 3 h at room temperature. Non-bound phages are removed and wells are washed extensively with PBS. For elution of specifically bound phages two different strategies are employed. Phages are either eluted using trypsin (1 mg/ml) for 15 min at room temperature or eluted first using an excess of a competitor such as purified soluble Nanobody 238D4 followed by trypsin elution as described before. Eluted phages are rescued and reamplified in E. coli TG1 for the next round of selection.

Selection outputs are analyzed for enrichment factors (# phages in eluate relative to controls) and outputs with highest enrichment factors are chosen for further analysis. For this the polyclonal phage pool is rescued in E. coli TG1 and E. coli cells are plated onto agar plates.

Individual TG1 colonies are picked and used in a PCR based screen.

### Example 2.5

### Screening of Nanobody-Fusion Proteins Via PCR

A PCR-based screening approach is employed to discriminate between clones 238D4 and 238D2 and unrelated clones at the N-terminal position of the Nanobody-fusion protein. In short, an equimolar mix of forward primers specific for the CDR3 sequence of either 238D4 or 238D2 is used in combination with a gIII-specific reverse primer. In case the Nanobody-fusion contains 238D2 or 238D4 at the N-terminal position the PCR yields two products with different lengths based on the two annealing sites for the CDR3-specific primers at the N-terminal (238D2 or 238D4) and central position (238D4). In case an unrelated Nanobody clone is present at the N-terminal position only the latter DNA fragment gets amplified.

Clones other than 238D4 and 238D2 are PCR amplified and PCR products are sequenced. Unique functional Nanobody clones are inserted into E. coli expression vector pAX100 as described above for further analysis.

### Example 2.6

### Evaluation of Binding Characteristics of Nanobodies

Individual Nanobodies different from 238D4 and 238D2 are assessed for specific binding to CXCR4. For this, periplasmic extracts are prepared and added to mictotiter plate wells coated with CXCR4+ and CXCR4− lipoparticles. As positive controls periplasmic extracts of 238D4 and 238D2 are used. Bound Nanobodies are detected with mouse anti-myc followed by rabbit anti-mouse-HRP and TMB.

Specific binders are further characterized in
a) Epitope binning experiments against 238D4 and 238D2
b) Ligand competition assays
c) Kinetic analysis
d) Functional cell-based assays to determine potency of monomeric and multimeric constructs

### Example 3

### Alternative Ways of Presenting the Antigen of Interest for Use in Immunization and/or Selection

A cell-free protein expression of membrane proteins using nanolipoprotein particles (e.g. MembraneMax system of Invitrogen) can also be used as an example of an in vitro translation system to express multi-membrane spanning proteins such as the antigens of the invention, e.g. complex targets such as e.g. GPCRs and ion channels.

Generation of nanolipoprotein particles—The coding sequence of the mature proteins is inserted in the pEXP5-CT/TOPO or another T7-based expression vector that allows expression of non-tagged or tagged (e.g. His-tag, Flag-tag) proteins. A C-terminal fusion of the protein with a Flag-tag (DYKDDDDK) is performed to allow purification, QC and detection of the protein.

Expression of the proteins in the in vitro translation system is performed as described in the kit manual (Invitrogen). The obtained material is analysed via SDS-PAGE and Western blot. Affinity purification using an anti-flag antibody bound to a resin could be used when the purity of the material is not sufficient.

Binding of target-specific antibodies and/or a ligand could be done as quality control of the produced proteins. The proteins could be captured on anti-flag tag coated beads, incubated with target-specific antibodies or a ligand (direct labeled or detection via antibody-fluorochrome) and measured in FACS.

Next to using this in vitro translation system, one can also make use of the baculovirus to produce viral-like particles and or membranes that over-expresses the antigen of interest, i.e. GPCR, ion channel. Using a dedicated expression vector, the GPCR/ion channel can be expressed on the membrane of baculovirus. The expression cassette used for expression of the TM-protein is such that a biotinylation site is included in the protein. As such only the protein of interest is biotinylated and can be detected/pulled down with labeled-SA or SA-coated beads. The baculovirus can be easily concentrated after culture by centrifugation and stored at 4° C. When used for panning, the virus pool can be treated with a mild detergent and the crude extract incubated with phages. Antigen-binding phages are then captured via SA-coated beads, washed and then eluted.

Upon selection for GPCR/ion channel binding Nanobodies/B-cells, these can be selectively recovered from the pool by streptavidin-coated beads.

1. An immunoglobulin single variable domain that specifically binds to the second extracellular loop of human CXCR4,
wherein the immunoglobulin single variable domain is not 238D2 (SEQ ID NO: 2) or 238D4 (SEQ ID NO: 3).

2. An immunoglobulin single variable domain that is a CXCR4 epitope A binder,
wherein the immunoglobulin single variable domain is not 238D2 (SEQ ID NO: 2).

3. An immunoglobulin single variable domain that is a CXCR4 epitope B binder,
wherein the immunoglobulin single variable domain is not 238D4 (SEQ ID NO: 3).

4. A polypeptide comprising the immunoglobulin single variable domain of claim 2.

5. The polypeptide of claim 4, further comprising the immunoglobulin single variable domain of claim 3.

6. The polypeptide of claim 4, further comprising a half-life extending moiety.

7. The polypeptide of claim 4, further comprising a linker.

8. The polypeptide of claim 4, wherein the polypeptide is selected from the group consisting of:
a. [CXCR4 epitope A binder]-linker-[CXCR4 epitope B binder];
b. [CXCR4 epitope A binder]-linker-[non-CXCR4 epitope B binder];
c. [CXCR4 epitope A binder]-linker-[CXCR4 epitope B binder];
d. [CXCR4 epitope A binder]-linker-[Nanobody/VHH binding to serum albumin]-linker-[CXCR4 epitope B binder];
e. [CXCR4 epitope A binder]-linker-[Nanobody/VHH binding to serum albumin]-linker-[non-CXCR4 epitope B binder];
f. [CXCR4 epitope A binder]-linker-[CXCR4 epitope B binder]-linker-[Nanobody/VHH binding to serum albumin];
g. [CXCR4 epitope A binder]-linker-[non-CXCR4 epitope B binder]-linker-[Nanobody/VHH binding to serum albumin];
h. [serum albumin]-linker-[CXCR4 epitope A binder]-linker-[CXCR4 epitope B binder];
i. [CXCR4 epitope A binder]-linker-[CXCR4 epitope B binder]-linker-[serum albumin];
j. [serum albumin binding peptide]-[CXCR4 epitope A binder]-linker-[CXCR4 epitope B binder];
k. [serum albumin binding peptide]-[CXCR4 epitope A binder]-linker-[non-CXCR4 epitope B binder];
l. [CXCR4 epitope A binder]-linker-[CXCR4 epitope B binder]-[serum albumin binding peptide];
m. [CXCR4 epitope A binder]-linker-[non-CXCR4 epitope B binder]-[serum albumin binding peptide]; and,
n. [CXCR4 epitope A binder]-linker-[CXCR4 epitope B binder], wherein the polypeptide is pegylated.

9. (canceled)

10. (canceled)

11. (canceled)

12. An antibody or fragment thereof that binds to D187, F189, E179 and S178 of human CXCR4 (SEQ ID NO: 6) and wherein said antibody or fragment thereof is an inverse agonist of said human CXCR4,
wherein said antibody or fragment thereof is not 238D4 (SEQ ID NO: 3), L3 (SEQ ID NO: 4) or L8 (SEQ ID NO: 5).

13. An antibody or fragment thereof that binds to F189, N192, W195, P191, V196, E277 of human CXCR4 (SEQ ID NO: 6) and wherein said antibody or fragment thereof is a neutral antagonist of said human CXCR4,
wherein said antibody or fragment thereof is not 238D2 (SEQ ID NO: 2).

14. An immunoglobulin single variable domain that binds to D187, F189, E179 and S178 of human CXCR4 (SEQ ID NO: 6) and wherein said immunoglobulin single variable domain is an inverse agonist of said human CXCR4,
wherein said immunoglobulin single variable domain is not 238D4 (SEQ ID NO: 3), L3 (SEQ ID NO: 4) or L8 (SEQ ID NO: 5).

15. An immunoglobulin single variable domain that binds to F189, N192, W195, P191, V196, E277 of human CXCR4 (SEQ ID NO: 6) and wherein said immunoglobulin single variable domain is a neutral antagonist of said human CXCR4,
wherein said immunoglobulin single variable domain is not 238D2 (SEQ ID NO: 2).

16. A pharmaceutical composition comprising the immunoglobulin single variable domain, or a polypeptide comprising the immunoglobulin single variable domain, of claim 1 and a pharmaceutically acceptable excipient.

17. A pharmaceutical composition comprising the immunoglobulin single variable domain, or a polypeptide comprising the immunoglobulin single variable domain, of claim 2 and a pharmaceutically acceptable excipient.

18. A pharmaceutical composition comprising the immunoglobulin single variable domain, or a polypeptide comprising the immunoglobulin single variable domain, of claim 3 and a pharmaceutically acceptable excipient.

19. The polypeptide of claim 8, wherein the Nanobody/VHH binding to serum albumin is Alb-11 (SEQ ID NO:17).

20. A polypeptide comprising the immunoglobulin single variable domain of claim 3.

21. The polypeptide of claim 20, further comprising a half-life extending moiety.

22. The polypeptide of claim 20, further comprising a linker.

23. The polypeptide of claim 20, wherein the polypeptide is selected from the group consisting of:
a. [CXCR4 epitope A binder]-linker-[CXCR4 epitope B binder];
b. [non-CXCR4 epitope A binder]-linker-[CXCR4 epitope B binder];
c. [CXCR4 epitope A binder]-linker-[CXCR4 epitope B binder];
d. [CXCR4 epitope A binder]-linker-[Nanobody/VHH binding to serum albumin]-linker-[CXCR4 epitope B binder];
c. [non-CXCR4 epitope A binder]-linker-[Nanobody/VHH binding to serum albumin]-linker-[CXCR4 epitope B binder];
d. [CXCR4 epitope A binder]-linker-[CXCR4 epitope B binder]-linker-[Nanobody/VHH binding to serum albumin];
e. [non-CXCR4 epitope A binder]-linker-[CXCR4 epitope B binder]-linker-[Nanobody/VHH binding to serum albumin];
f. [serum albumin]-linker-[CXCR4 epitope A binder]-linker-[CXCR4 epitope B binder];
g. [CXCR4 epitope A binder]-linker-[CXCR4 epitope B binder]-linker-[serum albumin];
h. [serum albumin binding peptide]-[CXCR4 epitope A binder]-linker-[CXCR4 epitope B binder];
i. [serum albumin binding peptide]-[non-CXCR4 epitope A binder]-linker-[CXCR4 epitope B binder];
j. [CXCR4 epitope A binder]-linker-[CXCR4 epitope B binder]-[serum albumin binding peptide];
k. [non-CXCR4 epitope A binder]-linker-[CXCR4 epitope B binder]-[serum albumin binding peptide]; and,
l. [CXCR4 epitope A binder]-linker-[CXCR4 epitope B binder], wherein the polypeptide is pegylated.

24. The polypeptide of claim 23, wherein the Nanobody/VHH binding to serum albumin is Alb-11 (SEQ ID NO:17).

25. A polypeptide comprising the immunoglobulin single variable domain of claim 14.

26. A polypeptide comprising the immunoglobulin single variable domain of claim 15.


SEQUENCE LISTING (30 sequences)

SEQ ID NO: 1 - protein, 360 aa, Homo sapiens [sequence omitted]

SEQ ID NO: 2 - protein, 122 aa, Artificial
  Nanobody
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSS

SEQ ID NO: 3 - protein, 128 aa, Artificial
  Nanobody
EVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 4 - protein, 265 aa, Artificial
  Nanobody construct
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSGGGGSGGGGSGGGGSEVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 5 - protein, 270 aa, Artificial
  Nanobody construct
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSGGGGSGGGGSGGGGSGGGGSEVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 6 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 7 - protein, 5 aa, Artificial
  Linker
GGGGS

SEQ ID NO: 8 - protein, 7 aa, Artificial
  Linker
SGGSGGS

SEQ ID NO: 9 - protein, 9 aa, Artificial
  Linker
GGGGSGGGS

SEQ ID NO: 10 - protein, 10 aa, Artificial
  Linker
GGGGSGGGGS

SEQ ID NO: 11 - protein, 15 aa, Artificial
  Linker
GGGGSGGGGSGGGGS

SEQ ID NO: 12 - protein, 18 aa, Artificial
  Linker
GGGGSGGGGSGGGGGGGS

SEQ ID NO: 13 - protein, 20 aa, Artificial
  Linker
GGGGSGGGGSGGGGSGGGGS

SEQ ID NO: 14 - protein, 25 aa, Artificial
  Linker
GGGGSGGGGSGGGGSGGGGSGGGGS

SEQ ID NO: 15 - protein, 30 aa, Artificial
  Linker
GGGGSGGGGSGGGGSGGGGSGGGGSGGGGS

SEQ ID NO: 16 - protein, 35 aa, Artificial
  Linker
GGGGSGGGGSGGGGSGGGGSGGGGSGGGGSGGGGS

SEQ ID NO: 17 - protein, 115 aa, Artificial [sequence omitted]

SEQ ID NO: 18 - protein, 23 aa, Artificial
  Synthetic peptide
AAAEQKLISEEDLNGAAHHHHHH

SEQ ID NO: 19 - protein, 352 aa, Artificial
  Mutant polypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRVYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 20 - protein, 352 aa, Artificial
  Mutant polypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWEVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 21 - protein, 352 aa, Artificial
  Mutant polypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICVRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 22 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 23 - protein, 122 aa, Artificial
  Nanobody
EVQLVESGGGLVQPGGSLRLSCAASGFTFSSYAMSWVRQAPGKGLEWVSGIKSSGDSTRYAGSVKGRFTISRDNAKNTLYLQMNSLRPEDTAVYYCAKSRVSRTGLYTYDNRGQGTLVTVSS

SEQ ID NO: 24 - protein, 128 aa, Artificial
  Nanobody
EVQLVESGGGLVQPGGSLRLSCAASGRTFNNYAMGWFRQAPGKEREFVAAITRSGVRSGVSAIYGDSVKDRFTISRDNAKNTLYLQMNSLRPEDTAVYYCAASAIGSGALRRFEYDYSGQGTLVTVSS

SEQ ID NO: 25 - protein, 128 aa, Artificial
  Nanobody
EVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 26 - protein, 122 aa, Artificial
  Nanobody
EVQLVESGGGLVQTGGSLRLSCAASGFTFSSYAMSWVRQAPGKGLEWVSGIKSSGDSTRYAGSVKGRFTISRDNAKNTLYLQMYSLKPEDTAVYYCAKSRVSRTGLYTYDNRGQGTQVTVSS

SEQ ID NO: 27 - protein, 270 aa, Artificial
  Nanobody construct
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSGGGGSGGGGSGGGGSGGGGSEVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 28 - protein, 270 aa, Artificial
  Nanobody construct
EVQLVESGGGLVQTGGSLRLSCAASGFTFSSYAMSWVRQAPGKGLEWVSGIKSSGDSTRYAGSVKGRFTISRDNAKNTLYLQMYSLKPEDTAVYYCAKSRVSRTGLYTYDNRGQGTQVTVSSGGGGSGGGGSGGGGSGGGGSEVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 29 - skipped

SEQ ID NO: 30 - protein, 270 aa, Artificial
  Nanobody construct
EVQLVESGGGLVQPGGSLRLSCAASGFTFSSYAMSWVRQAPGKGLEWVSGIKSSGDSTRYAGSVKGRFTISRDNAKNTLYLQMNSLRPEDTAVYYCAKSRVSRTGLYTYDNRGQGTLVTVSSGGGGSGGGGSGGGGSGGGGSEVQLVESGGGLVQPGGSLRLSCAASGRTFNNYAMGWFRQAPGKEREFVAAITRSGVRSGVSAIYGDSVKDRFTISRDNAKNTLYLQMNSLRPEDTAVYYCAASAIGSGALRRFEYDYSGQGTLVTVSS